SEQUENCE LISTING (2 sequences)

SEQ ID NO: 1 - DNA, 10 bp, Artificial Sequence
  Fluorescent quencher strand
gcatcggatc

SEQ ID NO: 2 - DNA, 20 bp, Artificial Sequence
  SNP discriminating loop primer (SD-LP)
gatccgatgcagtccgagtc

(19) United States
(12) Patent Application Publication
(10) Pub. No.: US 2016/0289752 A1
(43) Pub. Date: Oct. 6, 2016

(54) Method for the Isothermic Amplification of Nucleic Acid and SNP Detection

(71) Applicant: TRANSPONICS, ESSEX, VT (US)

(72) Inventor: Richard Bamford, Essex, VT (US)

(21) Appl. No.: 14/844,860

(22) Filed: Sep. 3, 2015

Related U.S. Application Data
(60) Provisional application No. 62/140,804, filed on Mar. 31, 2015.

(51) Int. Cl. C12Q 1/68

(52) U.S. Cl. CPC C12Q 1/6858

(57) ABSTRACT

The invention relates to a novel buffer formulation that is able to reduce reaction time compared to conventional LAMP buffer and may be universally applied to other LAMP reactions with little optimization required. The invention also relates to a modified LAMP method making use of the novel buffer and may incorporate SNP-discriminating forward loop primers to enhance the LAMP reaction while also reducing the likelihood of false-positives.

### CROSS-REFERENCE TO RELATED APPLICATIONS

This application is entitled to priority to U.S. Provisional Application No. 62/140,804, filed Mar. 31, 2015, which is incorporated herein by reference in its entirety.

### BACKGROUND OF THE INVENTION

Loop-mediated isothermal amplification (LAMP) is a simple, rapid, specific, and cost-effective nucleic acid amplification method when compared to PCR, nucleic acid sequence-based amplification, self-sustained sequence replication, and strand displacement amplification. The method generally employs a DNA polymerase and a set of four specially designed primers that recognize a total of six distinct sequences on a target DNA.

Conventional LAMP reactions rely on auto-cycling strand displacement DNA synthesis, which is carried out at 60-65° C. for 45-60 minutes in the presence of Bst DNA polymerase, dNTPs, two inner primers, two outer primers, and a target DNA template. The inner primers are called the forward inner primer (FIP) and the backward inner primer (BIP), and each contains two distinct sequences corresponding to the sense and anti-sense sequences of the target DNA. One inner primer initiates the LAMP reaction and the other is used for self-priming later stages.

After initiation by an inner primer, the pair of outer primers displaces the amplified strand with the help of Bst DNA polymerase. Bst DNA polymerase, having a high displacement activity, releases a single-stranded DNA that forms a hairpin to initiate the starting loop for cyclic amplification. The starting loop serves as a template for DNA synthesis primed by the second inner and outer primers that hybridize to the other ends of the target to produce a stem-loop DNA structure. In subsequent LAMP cycling, one inner primer hybridizes to the loop on the product and initiates displacement DNA synthesis to yield the original stem-loop DNA and a new stem-loop DNA with a stem that is twice as long. Amplification then proceeds in a cyclical order, where each strand is displaced during elongation with the addition of new loops with each cycle.

The present invention improves upon the conventional LAMP method to enhance the reaction rate and to reduce the likelihood of false-positives during SNP and mutation detection.

### SUMMARY OF THE INVENTION

The present invention relates to a novel buffer formulation for reducing reaction time compared to conventional LAMP buffer and a modified LAMP method using the same.

In one aspect, the invention relates to a buffer for isothermic amplification of nucleic acid. The buffer comprises 45 mM Tris-HCl at pH 7.75-8.0; 25 mM KCl; 25 mM (NH₄)₂SO₄; 0.2-0.25 mM dNTP; 1-8 units Bst DNA polymerase, large fragment; 550-825 nM Forward Inner Primer (FIP); and 550-825 nM Backward Inner Primer (BIP).

In one embodiment, the buffer further comprises 4 mM MgSO₄. In one embodiment, the buffer further comprises 3 mM MgCl₂. In one embodiment, the buffer further comprises an enhancer. In one embodiment, the enhancer is 2%-4% DMSO. In another embodiment, the enhancer is 1× solution of 0.6 M betaine and 2% DMSO. In one embodiment, the 1× solution of 0.6 M betaine and 2% DMSO is added at 0.5×.

In another aspect, the invention relates to a method of performing a modified LAMP reaction. The method comprises the steps of: preparing on ice a reaction mixture comprising target nucleic acid and 1× buffer of the present invention; heating the reaction mixture at 60° C.; returning the reaction mixture to ice; and detecting the modified LAMP reaction products.

In one embodiment, the reaction mixture is heated for 15-20 minutes. In one embodiment, the reaction mixture additionally comprises a SNP-discriminating forward loop primer (SD-LP). In one embodiment, the modified LAMP reaction products are detected by fluorescence. In one embodiment, the reaction mixture further comprises one or more primers selected from the group consisting of a back loop primer (BLP) and a forward loop primer (FLP).

In another aspect, the invention relates to a kit for performing isothermic amplification of nucleic acid, wherein the kit comprises a composition containing the buffer of the present invention. In one embodiment, the kit further comprises instructional material for performing the modified LAMP reaction method of the present invention.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1F depicts the progression of the SNP/LAMP reaction using SNP/LAMP buffer without outer primers.

FIG. 2 depicts the results of a SNP/LAMP experiment assessing the pathogenic Factor-V Lieden (FVL) A-allele, rs6025, with 2× pH 7.9 Klentaq buffer and varying Mg⁺⁺ concentrations with gDNA from P1 (a patient homozygous A/A).

FIG. 3 depicts the results of a SNP/LAMP experiment assessing the gDNA of multiple patients for the FVL mutant A-allele, rs6025, using 1.75× the standard amount of pH 7.9 Klentaq buffer concentration mixed 1:1 with ThermoPol II buffer without Mg.

FIG. 4 depicts the results of a SNP/LAMP experiment assessing the gDNA of multiple patients for the FVL wild-type G-allele, rs6025, with the same buffer as FIG. 3.

FIG. 5 depicts the results of a SNP/LAMP experiment assessing the A-allele and the C-allele of the chemokine binding protein 2 (CCBP2), rs2228468, with varying MgCl₂ concentrations, the gDNA from P3 (a patient homozygous A/A), and the same buffer as FIG. 3.

FIG. 6 depicts the results of a SNP/LAMP experiment assessing the A-allele and the C-allele of CCPB2, rs2228468, with varying MgCl₂ concentrations, the gDNA from P2 (a patient homozygous C/C), and the same buffer as FIG. 3.

FIG. 7 depicts the results of a SNP/LAMP experiment assessing the G-allele and the A-allele of ApoA5 SNP, rs10750097, with varying MgCl₂ concentrations, of the gDNA from P4 (a patient homozygous A/A), and the same buffer as FIG. 3.

FIG. 8 depicts results of a SNP/LAMP experiment assessing the reaction rate of the ApoA5 A-allele, rs10750097, with gDNA from P2 (a patient homozygous for A/A), and the same buffer as FIG. 3.

FIG. 9 depicts the results of a SNP/LAMP experiment assessing the minute-by-minute reaction rate of the ApoA5 A-allele, rs10750097, with gDNA from P2 and the same buffer as FIG. 3.

FIG. 10 depicts the results of a SNP/LAMP experiment assessing the reaction rate of the CCBP2 C-allele, rs2228468, with gDNA from P2 and the same buffer as FIG. 3.

FIG. 11 depicts the results of a SNP/LAMP experiment assessing the sensitivity of the ApoA5 A-allele, rs10750097, with cell lysate dilutions from P2 and the same buffer as FIG. 3.

FIG. 12 depicts the results of a SNP/LAMP experiment assessing the effect of increasing amounts of Tris-HCl pH 8.0 on the ApoA5 G-allele (rs10750097) reaction after 30 minutes incubation with 10 mM KCl, 10 mM (NH₄)₂SO₄, 0.2% TritonX-100, 3 mM MgCl₂ and P6 gDNA (a patient homozygous G/G).

FIG. 13 depicts the results of a SNP/LAMP experiment assessing the effect of increasing amounts of KCl on the ApoA5 G-allele (rs10750097) reaction after 25 minutes incubation with 45 mM Tris-HCl pH 8.0, 10 mM (NH₄)₂SO₄, 0.2% TritonX-100, 3 mM MgCl₂ and P6 gDNA.

FIG. 14 depicts the results of a SNP/LAMP experiment assessing the effect of increasing amounts of (NH₄)₂SO₄ on the ApoA5 G-allele (rs10750097) reaction after 25 minutes incubation with 45 mM Tris-HCl pH 8.0, 25 mM KCl, 0.2% TritonX-100, 3 mM MgCl₂, and P6 gDNA.

FIG. 15 depicts the results of a SNP/LAMP experiment assessing the effect of increasing amounts of TritonX-100 on the ApoA5 G-allele (rs10750097) reaction after 25 minutes incubation with 45 mM Tris-HCl pH 8.0, 25 mM KCl, 25 mM (NH₄)₂SO₄, 3 mM MgCl₂, and P6 gDNA.

FIG. 16 depicts the results of a SNP/LAMP experiment assessing the reaction time course of the ApoA5 A-allele with the final derivation of a 1×SNP/LAMP buffer, 0.25 mM dNTPs and 0.825 μM forward and reverse primers, 0.7 μL buccal cell gDNA (QE solution) and 3.2 units of Bst DNA polymerase in 10 μL reaction volumes.

FIG. 17 depicts the reaction rate in 2×SNP/LAMP buffer of a non-allelic amplicon that flanks the CCBP2 SNP, rs2228468.

FIG. 18 depicts an allelic assessment of the ApoA5 A-allele and G-allele, rs10750097, in 1×SNP/LAMP buffer with P6 gDNA.

FIG. 19 depicts an allelic assessment of the ApoA5 A-allele and G-allele, rs10750097, in 1×SNP/LAMP buffer with P2 gDNA.

FIG. 20 depicts the results of DMSO addition to enhance the rate of the ApoA5 G-allele (rs10750097) reaction with 1×SNP/LAMP buffer and P6 gDNA.

FIG. 21 depicts the results of DMSO addition at a threshold reaction time with the MyD88 wild-type allele (L275P; T>C) in 1×SNP/LAMP buffer with P2 gDNA.

FIG. 22 depicts the results of a titration of DMSO and betaine with the ApoA5 A-allele, rs10750097, in 1×SNP/LAMP buffer and P2 gDNA.

FIG. 23 depicts the time course comparison of reactions in 1×SNP/LAMP buffer (pH 7.75) vs. NEB's standard ThermoPol II buffer (pH 8.8) using the ApoA A-allele and P2 gDNA.

FIG. 24 depicts one embodiment of SNP/LAMP primer design with a theoretical example of a fluorescent SNP discriminating-loop primer (SD-LP).

FIG. 25 depicts the use of FLP, BLP, and SD-LP primers to enhance the SNP/LAMP reaction.

FIGS. 26A-26B depicts an example of SNP-detection using SNP/LAMP with a fluorescent SD-BLP assessing the C-allele of CCBP2, rs2228468, and gDNA from P2 and P3.

FIG. 27 depicts the analysis of familial gDNA for the pathogenic A-allele of FVL (rs6025) using SNP/LAMP with a fluorescent SD-BLP.

FIGS. 28A-28B depicts an example of miscopy analysis using SNP/LAMP with fluorescent SD-BLPs assessing the A-allele and the C-allele of CCBP2, rs2228468, with gDNA from P2 and P20 (a patient homozygous A/A).

FIG. 29 depicts an example of miscopy analysis using SNP/LAMP with a fluorescent SD-BLP assessing the pathogenic A-allele of FVL, rs6025, with gDNA from P2 and P21 (a patient heterozygous A/G).

FIGS. 30A-30C depicts a large scale allelic control study with a low frequency of miscopy for the ApoA5 A-allele and G-allele, rs10750097, using SNP/LAMP, a 3′ SD-FIP primer as the SNP discriminator, and gDNA from P2 and P6.

FIGS. 31A-31B depicts a high frequency of the miscopy phenomena using the same ApoA5 3′ SD-FIP primer as FIGS. 30A-30C and gDNA from P6.

FIGS. 32A-32B depicts a high frequency of the miscopy phenomena using 5′ SD-FIP and 5′ SD-BIP as SNP-discriminating primers for the ApoA5 G-allele, rs10750097 with P2 and P6 gDNA.

FIG. 33 depicts the allelic specificity for the MyD88 C/C amplicon (L265P; T>C) using a 3′ SD-FIP with gDNA from an ABC-DLBCL cell line (homozygous C/C) and a wild-type cell line (homozygous T/T).

FIG. 34 depicts a high frequency of the miscopy phenomena for the MyD88 C/C amplicon (L265P; T>C) using the same primers and gDNA as FIG. 33.

### DETAILED DESCRIPTION

The present invention is partly based upon the discovery that certain methods for the isothermic amplification of nucleic acids yield faster reaction rates and decrease the occurrence of false-positives. The results described herein demonstrate that a novel buffer formulation is able to reduce reaction time compared to conventional LAMP buffer, and can be universally applied to other LAMP reactions. The results also demonstrate that a modified LAMP method making use of the novel buffer is able to enhance the LAMP reaction and may incorporate SNP-discriminating forward loop primers to reduce the likelihood of false-positives.

### DEFINITIONS

It is to be understood that the figures and descriptions of the present invention have been simplified to illustrate elements that are relevant for a clear understanding of the present invention. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

An “allele” refers to one specific form of a genetic sequence (such as a gene) within a cell, an individual or within a population, the specific form differing from other forms of the same gene in the sequence of at least one, and frequently more than one, variant sites within the sequence of the gene. As used herein the terms “alteration,” “defect,” “variation,” or “mutation,” refers to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

As used herein, the term “wild-type” refers to a gene or gene product isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “amplification” refers to the operation by which the number of copies of a target nucleotide sequence present in a sample is multiplied.

The term “amplification reagents” as used herein relate to chemical or biochemical components that enable the amplification of nucleic acids. Such reagents comprise, but are not limited to, nucleic acid polymerases, buffers, mononucleotides such as nucleoside triphosphates, oligonucleotides e.g. as oligonucleotide primers, salts and their respective solutions, detection probes, dyes, and more.

“Simultaneously,” in the sense of the invention, means that two actions, such as amplifying a first and a second or more nucleic acids, are performed at the same time and under the same physical conditions. In one embodiment of the invention, simultaneous amplification of the at least first and second target nucleic acids is performed in one vessel. In another embodiment, simultaneous amplification is performed with at least one nucleic acid in one vessel and at least a second nucleic acid in a second vessel, at the same time and under the same physical conditions, particularly with respect to temperature and incubation time.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

The term “gene” refers to a nucleic acid (e.g., DNA) sequence that includes coding sequences necessary for the production of a polypeptide, precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids. A single molecule that contains pairing of complementary nucleic acids within its structure is said to be “self-hybridized.” A single DNA molecule with internal complementarity could assume a variety of secondary structures including loops, kinks or, for long stretches of base pairs, coils.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The phrase “biological sample” is used herein in its broadest sense. A sample may be of any biological tissue or fluid from which biomarkers of the present invention may be detected, extracted, isolated, characterized or measured. Examples of such samples include but are not limited to blood, lymph, urine, gynecological fluids, biopsies, amniotic fluid and smears. Samples that are liquid in nature are referred to herein as “bodily fluids.” Biological samples may be obtained from a patient by a variety of techniques including, for example, by scraping or swabbing an area or by using a needle to aspirate bodily fluids. Methods for collecting various biological samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biological samples also include tissues, such as, frozen sections taken for histological purposes. The sample also encompasses any material derived by processing a biological sample. Derived materials include, but are not limited to, cells (or their progeny) isolated from the sample, proteins or nucleic acid molecules extracted from the sample. Processing of a biological sample may involve one or more of: filtration, distillation, extraction, concentration, inactivation of interfering components, addition of reagents, and the like.

As used herein, the term “polymerase chain reaction” (“PCR”) refers to the method of K. B. Mullis (U.S. Pat. No. 4,683,195 4,683,202, and 4,965,188, hereby incorporated by reference) for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product,” “PCR fragment,” “amplification product” or “amplicon” refer to the resultant mixture of compounds after two or more cycles of the PCR steps of denaturation, annealing and extension are complete. These terms encompass the case where there has been amplification of one or more segments of one or more target sequences.

As used herein, the term “probe” refers to an oligonucleotide (i.e., a sequence of nucleotides), whether occurring naturally as in a purified restriction digest or produced synthetically, recombinantly or by amplification, that is capable of hybridizing to another oligonucleotide of interest. A probe may be single-stranded or double-stranded. Probes are useful in the detection, identification and isolation of particular gene sequences.

The term “perfect match,” “match,” “perfect match probe” or “perfect match control” refers to a nucleic acid that has a sequence that is perfectly complementary to a particular target sequence. The nucleic acid is typically perfectly complementary to a portion (subsequence) of the target sequence. A perfect match (PM) probe can be a “test probe,” a “normalization control” probe, an expression level control probe and the like. A perfect match control or perfect match is, however, distinguished from a “mismatch” or “mismatch probe.”

The term “mismatch,” “mismatch control” or “mismatch probe” refers to a nucleic acid whose sequence is not perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

The term “reaction mixture” or “master mix” or “master mixture” refers to an aqueous solution of constituents in an amplification reaction that can be constant across different reactions. An exemplary amplification reaction mixture includes buffer, a mixture of deoxyribonucleoside triphosphates, primers, probes, and DNA polymerase. Generally, template RNA or DNA is the variable in an amplification reaction.

As used herein, “purified” refers to being essentially free of other components. For example, a purified polypeptide is a polypeptide which has been separated from other components with which it is normally associated in its naturally occurring state.

The term “single nucleotide polymorphism” or “SNP” is a DNA sequence variation which occurs within the genome of an organism, wherein a single nucleotide base differs between members of a species. The DNA sequence variation usually results in a change in the single nucleotide base which is different from the expected nucleotide base at that position. The term “mutant allele” is used to refer to a change in the single nucleotide base from the sequence which is found in the majority of the species to an unexpected and different single nucleotide base not commonly found within the species. The term “wild type” is used to refer to the presence of the expected single nucleotide base which is found in the majority of the species.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Targets are sometimes referred to in the art as anti-probes. As the term “targets” is used herein, no difference in meaning is intended.

“Instructional material,” as that term is used herein, includes a publication, a recording, a diagram, or any other medium of expression which can be used to communicate the usefulness of the nucleic acid, peptide, and/or compound of the invention in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the nucleic acid, polypeptide, and/or compound of the invention or be shipped together with a container that contains the nucleic acid, polypeptide, and/or compound. Alternatively, the instructional material may be shipped separately from the container with the intention that the recipient uses the instructional material and the compound cooperatively.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

### SNP/LAMP Buffer

Herein is described a major improvement on the molecular diagnostic tool, loop-mediated isothermic amplification (LAMP) of DNA, originally described by Notomi, et al., 2000, Nucleic Acids Research 28(12):i-vii. The inventive composition, the Single Nucleotide Polymorphism/Loop-Mediated Isothermic Amplification (SNP/LAMP) buffer, makes several significant changes to the formulation of the conventional LAMP buffer that impacts the technology at several levels:

First, SNP/LAMP buffer greatly enhances the reaction rate a LAMP reaction without the use of outer primers.

Second, because of the universal nature of the SNP/LAMP buffer:

i. Primers are designed around a small temperature range.

ii. The optimization time of the primers is minimal.

iii. The maximal reaction times and temperatures are very similar, therefore allowing for multiple targets to be run simultaneously.

Third, the LAMP reactions use lower dNTP and primer concentrations than other published LAMP reactions, and the LAMP reactions are typically run at 54 volumes making them very cost efficient.

A conventional buffer preparation for a LAMP reaction comprises: at least one polymerase enzyme, wherein the enzyme is capable of strand displacement, a target-specific primer set, and dinucleotide triphosphates (dNTPs) in a single, dry format; wherein said reagent preparation is water soluble and stable above 4° C.

Any suitable DNA polymerase capable of strand displacement can be employed. As used herein, the term “strand displacement” refers to the ability of the enzyme to separate the DNA strands in a double-stranded DNA molecule during primer-initiated synthesis. The enzyme can be a complete enzyme or a biologically active fragment thereof. The enzyme can be isolated and purified or recombinant. In some embodiments, the enzyme is thermostable. Such an enzyme is stable at elevated temperatures (>40° C.) and heat resistant to the extent that it effectively polymerizes DNA at the temperature employed. Sometimes the enzyme can be only the active portion of the polymerase molecule, e.g., Bst large fragment. Exemplary polymerases useful in the methods of the invention include, but are not limited to Bst DNA polymerase, Vent DNA polymerase, Vent (exo-) DNA polymerase, Deep Vent DNA polymerase, Deep Vent (exo-) DNA polymerase, Bca (exo-) DNA polymerase, DNA polymerase I Klenow fragment, Φ29 phage DNA polymerase, Z-Taq™ DNA polymerase, ThermoPhi polymerase, 9° Nm DNA polymerase, and KOD DNA polymerase. See, e.g., U.S. Pat. Nos. 5,814,506; 5,210,036; 5,500,363; 5,352,778; and 5,834,285; Nishioka, M., et al. (2001) J. Biotechnol. 88, 141; Takagi, M., et al. (1997) Appl. Environ. Microbiol. 63, 4504.

The primers in a conventional LAMP buffer are target-specific. The target-specific primers are designed so that they permit the amplification of the target nucleotide sequence using the LAMP method. See, e.g., U.S. Pat. No. 6,410,278; U.S. Appl. No. 2006/0141452; and Nagamine et al., Clin. Chem. (2001) 47:1742-43. A primer, which is used for synthesizing the desired nucleic acid sequence, is not particularly limited in length as long as it complementarily binds as necessary.

The conventional LAMP buffer provides a pH that is suitable for the enzyme reaction, salts necessary for annealing or for maintaining the catalytic activity of the enzyme, a protective agent for the enzyme, and, as necessary, a regulator for melting temperature (Tm). An exemplary buffer is Tris-HCl, having a buffering action in a neutral to weakly alkaline range. The pH is adjusted depending on the DNA polymerase used. The salts, KCl, NaCl, (NH₄)₂SO₄, etc. are added to maintain the activity of the enzyme and to regulate the melting temperature (Tm) of nucleic acid. The protective agent for the enzyme makes use of bovine serum albumin or sugars. Further, dimethyl sulfoxide (DMSO) or formamide can be used as the regulator for melting temperature (Tm). By use of the regulator for melting temperature (Tm), annealing of the oligonucleotide can be regulated under limited temperature conditions. Further, betaine (N,N,N-trimethylglycine) or a tetraalkyl ammonium salt is also effective for improving the efficiency of strand displacement by virtue of its isostabilization. By adding betaine, its promoting action on the nucleic acid amplification of the present invention can be expected. Because these regulators for melting temperature act for lowering melting temperature, those conditions giving suitable stringency and reactivity are empirically determined in consideration of the concentration of salts, reaction temperature, etc.

Specific composition differences between conventional LAMP buffer (as described for HBV, HCV, PSA detection in U.S. Pat. No. 6,410,278) and one embodiment of SNP/LAMP buffer, are shown in Table 1.

TABLE 1

| Conventional | SNP/LAMP |
|---|---|
| LAMP Buffer (25 μL) | Buffer (adjusted to 25 μL) |
| 20 mM Tris-HCl, pH 8.8 | 45 mM Tris-HCl, pH 7.75 |
| 10 mM KCl | 25 mM KCl |
| 10 mM (NH₄)₂SO₄ | 25 mM (NH₄)₂SO₄ |
| 4 mM MgSO₄ | 3 mM MgCl₂ |
| 1M Betaine | 3% DMSO |
| 0.1% Triton X-100 | No detergent added |
| 0.4 mM dNTP | 0.25 mM dNTP |
| 8 units Bst DNA polymerase, large | 8 units Bst DNA polymerase, large |
| fragment* | fragment* |
| 1600 nM Forward Inner Primer | 825 nM Forward Inner Primer (FIP) |
| (FIP) | |
| 1600 nM Backward Inner Primer | 825 nM Backward Inner Primer (BIP) |
| (BIP) | |
| 400 nM Forward Outer Primer | Not used |
| 400 nM Backward Outer Primer | Not used |

*New England Biolabs, Inc.

TABLE 2
SNP/LAMP Components and Concentrations/Amounts

| Component | Concentration/Amount |
|---|---|
| Tris-HCl, pH 7.75-8.0 | 40-50 mM |
| KCl | 20-30 mM |
| (NH₄)₂SO₄ | 20-30 mM |
| MgSO₄/MgCl₂ | 3-5 mM |
| DMSO | 2-4% (or omitted) |
| Betaine | 0.6M (or omitted) |
| Triton X-100 | 0-0.2% |
| dNTP | 0.15-0.35 mM |
| Bst DNA polymerase, large fragment* | 2-10 units (per 25 μL) |
| Forward Inner Primer (FIP) | 550-825 nM |
| Backward Inner Primer (BIP) | 550-825 nM |
| 400 nM Forward Outer Primer | Not used |
| 400 nM Backward Outer Primer | Not used |

*New England Biolabs, Inc.

The presence of certain components and the concentrations of components that comprise the SNP/LAMP buffer may vary depending on the volume of fluid sample containing target nucleic acids. As a non-limiting example, the pH of Tris-HCl may be between pH 7.75 and pH 8.0. MgSO₄ may be interchangeable with MgCl₂. As a non-limiting example, the MgCl₂ concentration may be 3 mM MgCl₂. DMSO, as an enhancer, may be omitted. If DMSO is desired, it may be included, as a non-limiting example, between 2% and 4% DMSO. An alternative to DMSO as an enhancer is a 1× solution of 0.6M betaine and 2% DMSO. As a non-limiting example, dNTP may be between 0.2 mM and 0.25 mM. As a non-limiting example, Bst DNA polymerase may be between 1.6 units and 8 units. As a non-limiting example, FIP may be between 550 nM and 825 nM. As a non-limiting example, BIP may be between 550 nM and 825 nM.

One aspect of the invention is the SNP/LAMP buffer described above, wherein at least one fluid sample of said plurality of different fluid samples has a different volume than the other fluid samples. In one embodiment, alternatively or additionally, different volumes of SNP/LAMP buffer are added to said plurality of different fluid samples. In a further embodiment, when at least one fluid sample of said plurality of different fluid samples has a different volume than the other fluid samples, SNP/LAMP buffer is added to the samples such that all samples have the same volume after addition. The advantages of being able to choose an appropriate starting volume depending on the sample type, and of having identical volumes for carrying out the isolation and optionally, e.g., amplification and detection, are combined in this approach.

### Modified LAMP Method

For SNP/LAMP, the FIP and BIP primers are designed so that the template targeting, sense and anti-sense half, of each primer has a melting temperature (Tm) of 60° C. to 65° C. (based on the above magnesium, dNTP, and primer concentration). Additionally, no nucleotide spacers are used between the sense and anti-sense portions of the primers. For reaction quality and fidelity, it is essential to purify the FIP and BIP primers using HPLC after synthesis.

The SNP/LAMP reaction is set-up on ice and then the reaction tubes are transferred to a heat block or water bath at 60° C. Reactions are typically allowed to run for 30 minutes and then transferred to an ice block (4° C.). However, for most reactions, including single nucleotide polymorphism (SNP) detection, the product can be easily visualized by gel electrophoresis by 20 to 25 minutes when 200-400 target copies are used per 5 μL reaction. This includes genomic DNA isolated using rapid/crude extraction techniques such as QuickExtract Solution (Epicentre). Additionally, the rate can be further enhanced if loop primers (FLP and BLP) are present (FIG. 25). The addition of 3% DMSO to the SNP/LAMP reaction appears to increase the reaction rate and its specificity of most amplicons tested, some variation on the percentage of DMSO added may be considered depending on the G/C nature of the amplicon.

With the SNP/LAMP method, it is demonstrated that the double stranded (ds) DNA templates, such as genomic DNA (even crude extracts at low copy numbers), are more receptive to binding target-specific, single stranded (ss) DNA oligonucleotides (primers) at non-denaturing temperature conditions (e.g., 60° C.), as compared to conventional LAMP. The more “receptive” or perhaps “relaxed” state of dsDNA in the SNP/LAMP buffer may reflect changes in the kinetics of dynamic equilibrium and/or the ability of primers to strand invade. In turn, this results in greatly enhanced reaction rates, independent of outer primers. The method's reaction takes advantage of the thermophilic, strand displacing characteristics of Bst DNA polymerase (large fragment) and loop generating primers.

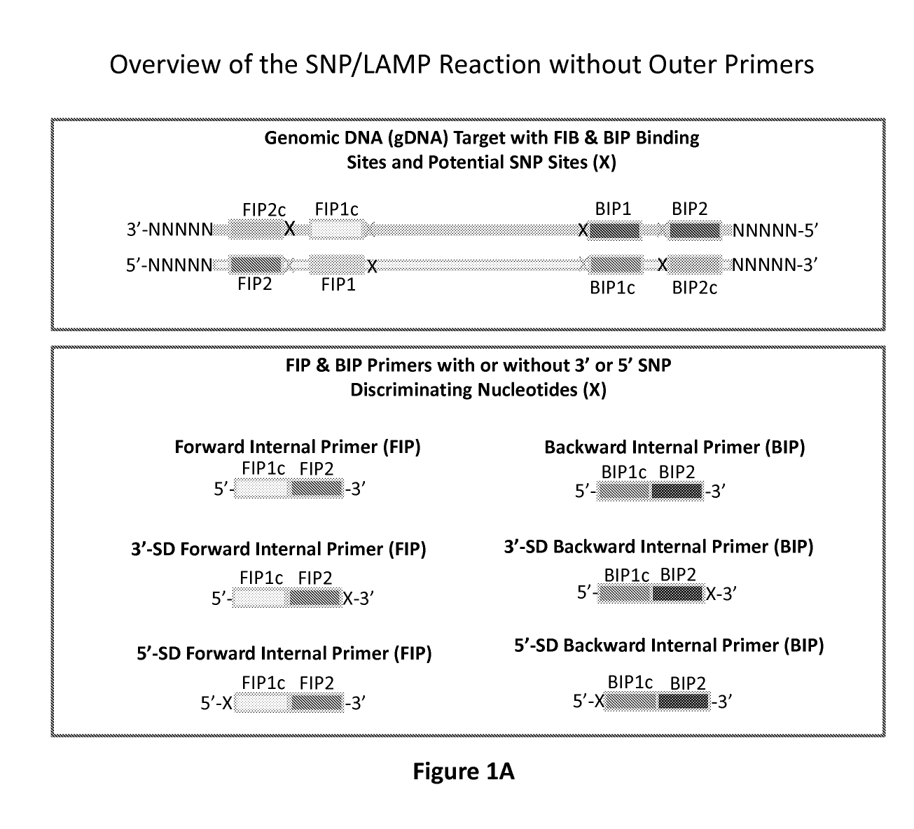

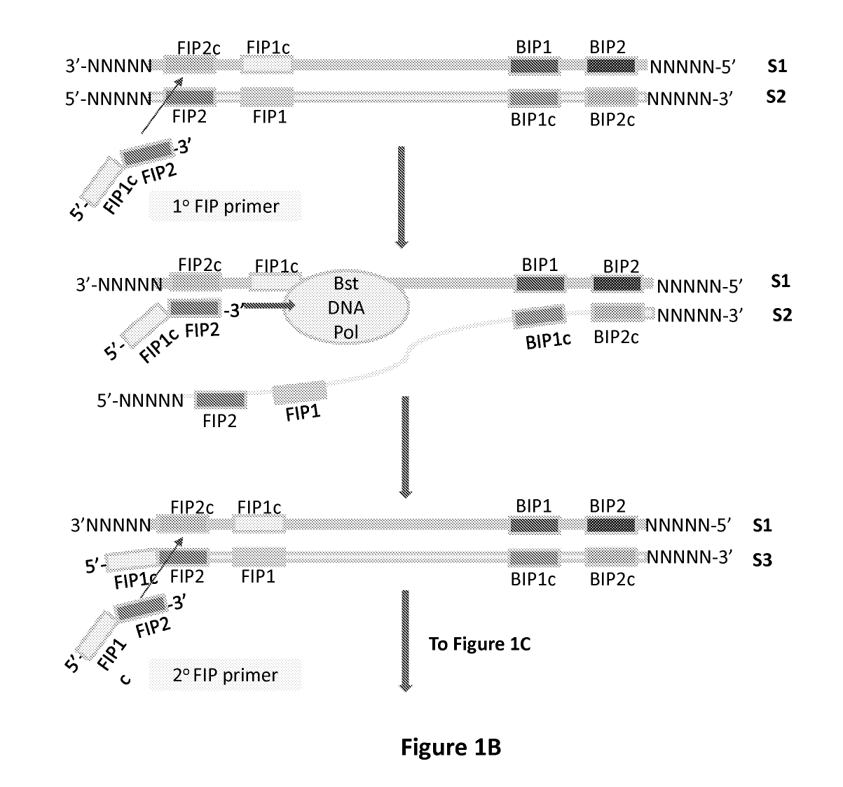

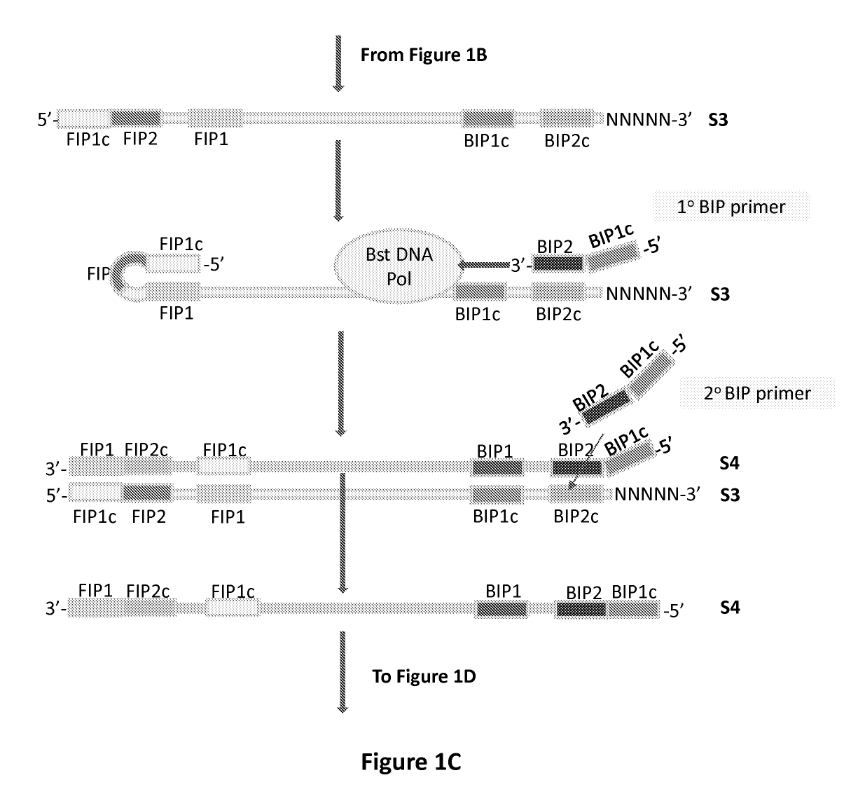

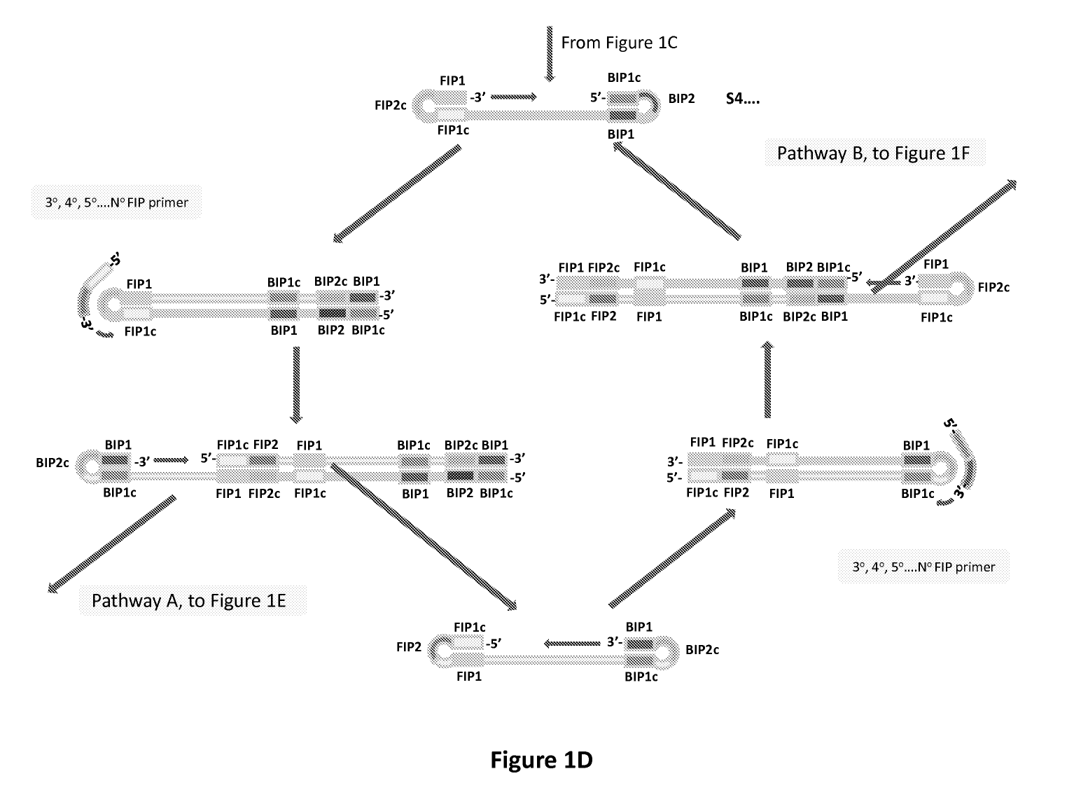

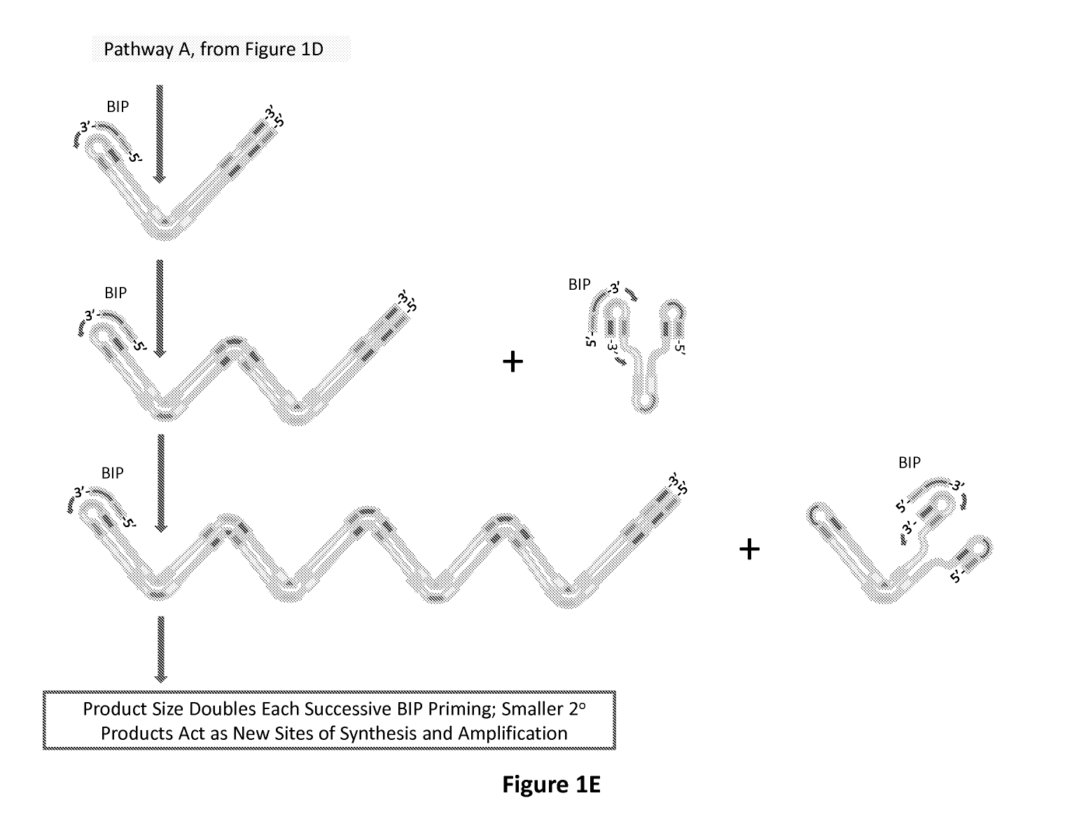

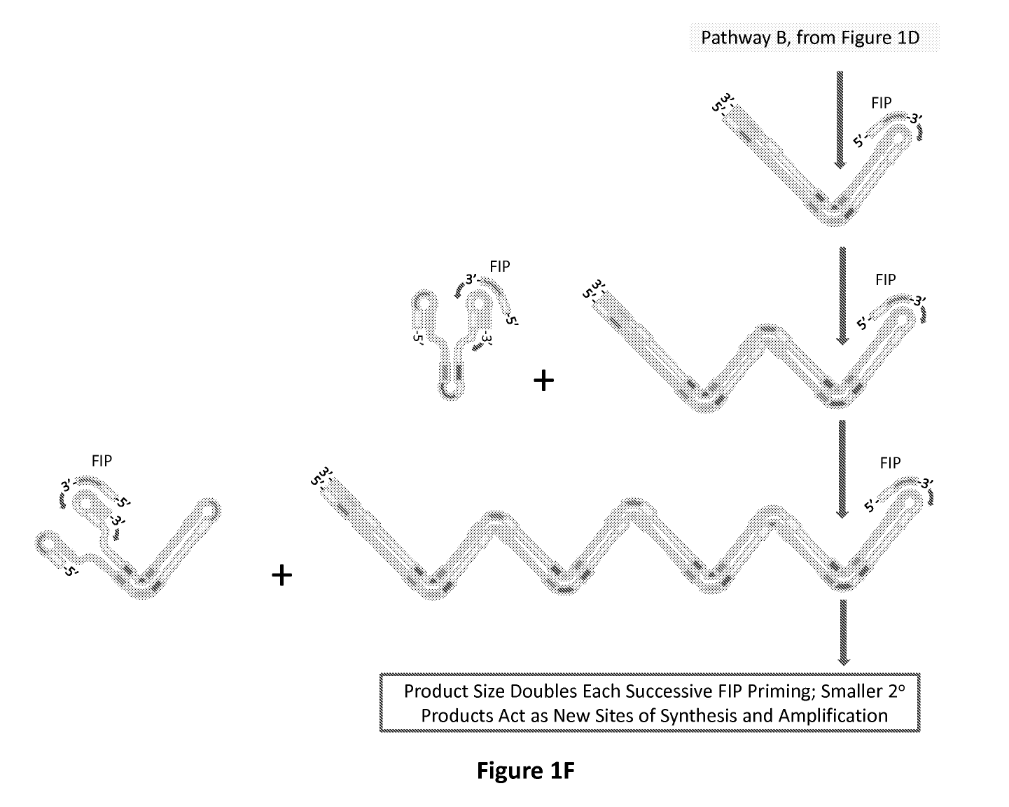

As an example of the method, an arbitrary, dsDNA with defined target sequences (FIG. 1A, top panel), is initially targeted via dynamic equilibrium and/or strand invasion by a synthetic, ssDNA primer (FIP or forward inner primer; FIG. 1A bottom panel and FIG. 1B). Here, the 3′ half of the FIP primer (FIP2) is complementary to the FIP2c-region of the dsDNA. Additionally, the FIP primer also contains a nested sequence in its 5′-half (FIP1c) that is identical to a region downstream of its original binding site (FIP2c). This design, as described elsewhere herein, ultimately allows for the loop-mediated amplification seen with LAMP and SNP/LAMP.

With the FIP2-region of FIP bound to FIP2c, the 3′-hydroxy (OH) end of the primer is extended (FIG. 1B) by Bst DNA polymerase, synthesizing a complementary strand (S3) of the target DNA while at the same time displacing the parental complementary strand (S2) of the target DNA. The displaced S2 strand now becomes the target for BIP and initiates the “reverse strand” reaction; however, for the sake of simplicity, only the “forward” reaction will be followed.

Under conditions that appear unique to the SNP/LAMP reaction (FIG. 1B), a second copy of FIP, via dynamic equilibrium and/or strand invasion, readily binds its respective target regions (FIP2c) on the dsDNA complex, S1/S3. This event, under conventional LAMP conditions, appears rate-limiting and as a result requires the use of outer primers (OPs) to displace the primary FIP and BIP primers. As the secondary FIP primer is extended, it displaces the S3 strand (FIG. 1C).

Of note, the 5′ FIP1c-region of S3 will loop back and bind to its complementary sequence (FIP1, on the same strand) forming a loop structure; however, since the 5′-phosphate group cannot be extended by DNA polymerase, the structure is inert. In its current ssDNA state, the BIP2c-region of S3 can now behave as a target for the BIP primer (FIG. 1C) which is then extended to the 5′-end of S3, generating the complementary S4 strand.

By the same mechanism described in FIG. 1B, now a second BIP primer binds the BIP2c-region of the S3/S4 dsDNA complex (FIG. 1C) and is extended, displacing the S4 strand. At this stage, single stranded S4 now has the 3′ and 5′ ends of the amplicon established (FIP1 and BIP1c, respectively).

The S4 strand (in its dumbbell-like structure) can now enter the cyclic amplification stage (FIG. 1D), which is perpetuated by new FIP and BIP primers on each side of the cycle. Reactions branching off the cycle are represented by Pathways A and B in FIGS. 1E and 1F, respectively. Pathway A (FIG. 1E) is driven by new copies of the BIP primer targeting its single-stranded, complementary loop structure. Each iterative binding and extension of a BIP primer doubles the product size and generates new secondary products, or “seed sequences,” which act as new sites of synthesis and amplification. All products grow as alternating inverted repeats, and continue to generate new seed sequences, resulting in an extremely rapid, exponential reaction. Pathway B (FIG. 1F) represents the reverse and complementary reaction to Pathway A, but it is driven by the FIP primer.

For SNP detection using FIP and/or BIP primers, the same amplification schematic would be followed. However, the reaction would not occur if the 3′ or 5′ SNP discriminating nucleotides, designed into either FIP or BIP primers (FIG. 1A, lower panel), do not complement the SNP being assayed.

### Kits of the Invention

The invention also includes a kit comprising compounds useful within the methods of the invention and an instructional material that describes, for instance, the method of using the SNP/LAMP buffer with the modified LAMP method as described elsewhere herein, or the method of using the SNP/LAMP buffer with other LAMP methods. In an embodiment, the kit further comprises (preferably sterile) the components of the SNP/LAMP buffer in premeasured amounts suitable for reconstitution and immediate use. Such kits can further include, in addition to the buffer, one or more additional component, such as reaction containers, and additional reagents such as amplification enzyme(s), primers, probes, sterilized water, lysis buffer, stop buffer, and the like.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Buffer Development for the SNP/LAMP System

One of the early prototypes of conventional LAMP was a PCR/LAMP hybrid system that used Klentaq1 Reaction Buffer (RB#20) or Klentaq Mutant Reaction Buffer (RB#10), from DNA Polymerase Technology, Inc.

1× Klentaq1 Reaction Buffer (Cat#RB20)
50 mM Tris-Cl pH 7.9
16 mM (NH₄)₂SO₄
3.5 mM MgCl₂
0.05% Brij 58 (detergent)

1× Klentaq Mutant Reaction Buffer (Cat#RB10)
50 mM Tris-Cl pH 7.9
16 mM (NH₄)₂SO₄
3.5 mM MgCl₂
0.025% Brij 58 (detergent)

Both buffers were offered with pH 9.2 Tris-C1, but it was suggested by the manufacturer that the allelic specificity of their polymerase (Klentaq) was greater at pH 7.9 (though it may slow reaction time); therefore, noting the desire to target SNPs, Klentaq's lower pH formulation was tested with the PCR/LAMP hybrid system. Surprisingly, Bst DNA Polymerase, large fragment (cat#M0275S, New England Biolabs (NEB)) appeared to work well in the above buffer conditions, despite the higher Tris-Cl pH 7.9 concentration (50 mM vs. 20 mM) and the lower pH (7.9 vs. 8.8), the higher (NH₄)₂SO₄ concentration (16 mM vs. 10 mM), and the absence of KCl (0 mM vs. 10 mM). These numbers are compared with the more conventional ThermoPol buffer, which is usually recommended for Bst DNA polymerase.

Standard 1× ThermoPol Reaction Buffer (Cat#B9004S, NEB)
20 mM Tris-Cl
10 mM (NH₄)₂SO₄
10 mM KCl
2 mM MgCl₂
0.1% Triton® X-100
pH 8.8 @ 25° C.

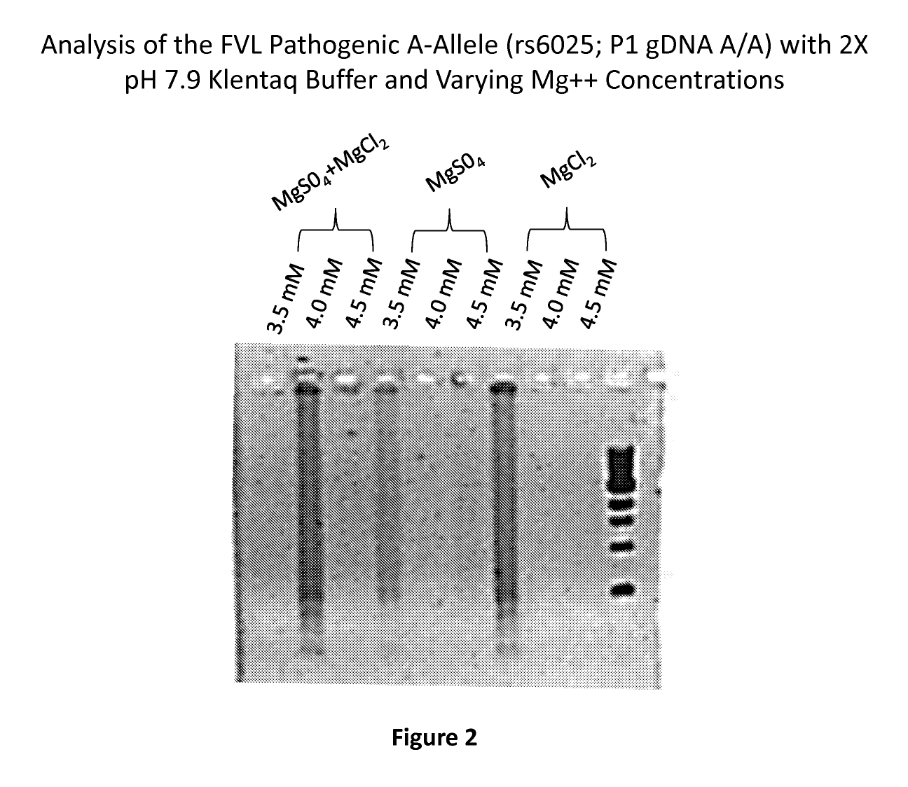

Two or three cycles of PCR were originally included in the SNP/LAMP reaction to eliminate the use of outer primers by creating the barbell, replicating species (see FIG. 1D, S4 strand) with only FIP and BIP primers. Unexpectedly, it was determined that with the non-standard Bst DNA polymerase buffer conditions (above and below) that the reaction was working well in the absence of outer primers and therefore the PCR step was eliminated. After removing the PCR component of the reaction, the pH 7.9 Klentaq buffer was used singly, or in combination (1:1) with the above ThermoPol buffer. By mixing the buffers, the KCl component of the buffer was added back and raised the pH slightly. To further optimize the SNP/LAMP reaction conditions, versions of Klentaq1 buffer (RB20) and ThermoPol buffer were purchased without a magnesium source (MgCl₂ and MgSO₄, respectively; ThermoPol buffer without MgSO₄ is sold by NEB as ThermoPol II Buffer, cat#B9005S, and Klentaq1 buffer without MgCl₂ was a special request). Subsequently, to assess the magnesium effect in isolation, different MgCl₂ and/or MgSO₄ concentrations were supplemented back to the reactions. Surprisingly, while doing a magnesium titration with the pH 7.9 Klentaq buffer for a SNP/LAMP amplicon targeting the Factor-V Lieden (FVL) pathogenic A-allele, rs6025, reaction products were observed when 2× the standard amount of buffer was inadvertently added to the reactions (FIG. 2); whereas no products were seen in similar experiments with 1× Klentaq buffer at the same time point. Other components of the 2× reaction included 0.8 uM forward and reverse primers, 0.2 mM dNTP (each), variable MgCl₂ or MgSO₄, 1 μL of buccal cell gDNA (from P1, a patient homozygous A/A for FVL) and 4.8 units of Bst DNA Polymerase in a 15 μL reaction volume. Reaction time was 1 hr at 60° C., and products here, and in subsequent studies (unless stated), were resolved on 1% TBE gels (75 parts agarose/25 parts Synergel), and were visualized and photo-documented with ethidium bromide staining and UV light.

Of note, for FIGS. 2 through 11, these experiments included a minute quantity of the heat labile nicking enzyme NbBbVC1 (NEB). In theory, it was felt that the enzyme would nick the target gDNA early (the enzyme would be rapidly inactivated at 60° C.) and these nick sites would be subsequently extended by Bst DNA Polymerase. As the nicks are extended, they displace single-stranded gDNA including the target sequence for the SNP/LAMP. This would, again in theory, allow for the FIP and BIP primers to target their respective sites more rapidly than dynamic equilibrium. However, subsequent, more thorough studies determined that the presence of the nicking enzyme had little or no effect on reaction initiation and it was ultimately dropped from the formulation.

Noting the 2× buffer concentration in the above reactions, other Klentaq buffer concentrations were tested and the concentration of 1.75× appeared optimal. Nevertheless, the initiation of these reactions and the subsequent generation of the products were not consistent and didn't seem to follow patterning relative to the magnesium concentrations. Therefore, to address this observed inconsistency, different combinations of the buffers were assessed including a 1.75× concentration of the Klentaq1 reaction buffer (no Mg) mixed 1:1 with ThermoPol II buffer (no Mg).

1.75× Klentaq1/ThermoPol II Buffer Mix (K1/TPIIB Mix)
61.25 mM Tris-HCl (combined ˜pH 8.125)
22.75 mM (NH₄)₂SO₄
8.75 mM KCl
0.0875% Brij 58 (detergent)
0.175% Triton® X-100
Mg concentrations varied depending on the allele being assessed

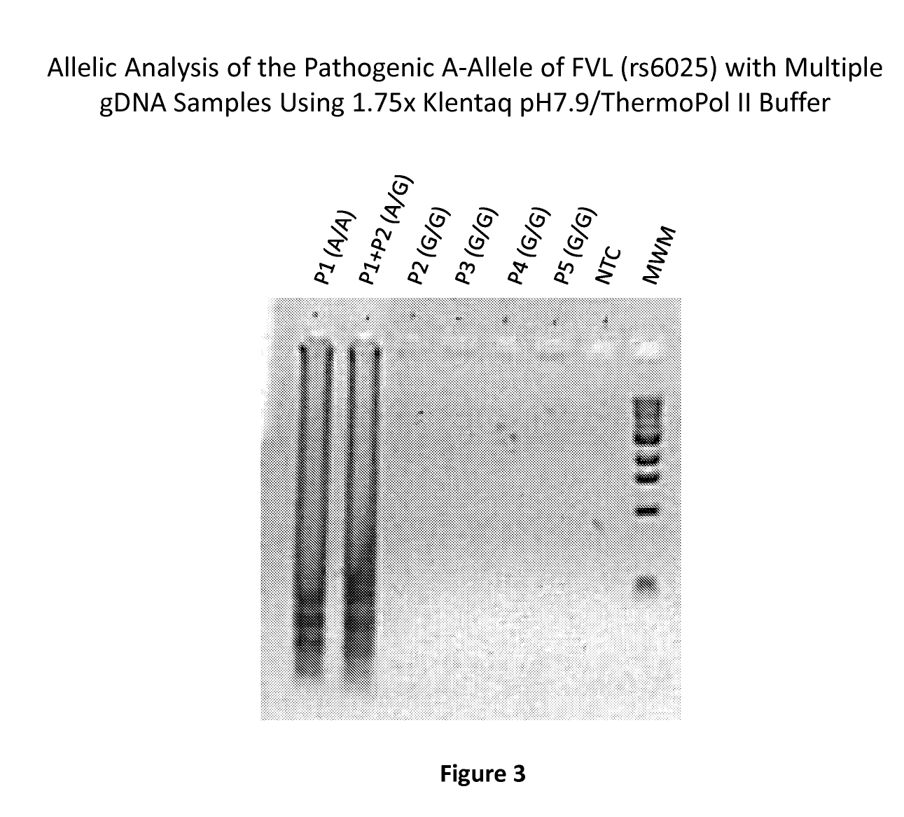

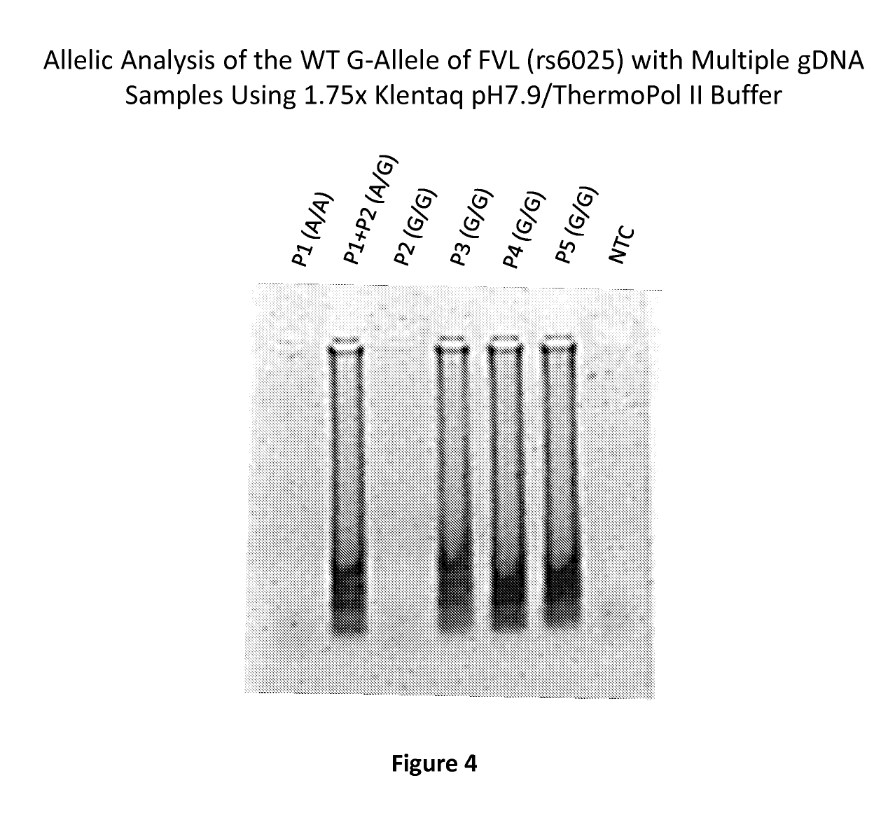

With this new buffer formulation, there was a demonstrable improvement in reaction initiation and product generation with SNP/LAMP. The ability of the reaction to discern specific alleles or SNPs for several different genes was also demonstrated with this buffer. This includes the Factor-V Lieden (FVL) allele (rs6025; G>A) which is strongly associated with thrombosis. FIG. 3, which assesses the pathogenic, mutant A-allele, shows a reaction product from the gDNA of an individual P1 who is homozygous for the trait (i.e., A/A). Since no heterozygous carrier was available, a pseudo (ψ) heterozygote (G/A) was generated by mixing the gDNA from P1 (A/A), 1 to 1, with the gDNA from P2, a known homozygous wild-type (G/G). The ψ-heterozygote (A/G), as predicted, also shows a strong reaction product. Conversely, P2, P3, P4, and P5, all known wild-types, show no reaction product, this is also true for the no template control (NTC). As a reciprocal study, the same gDNA samples were assessed for the wild-type, G-allele for the Factor-V gene, FIG. 4. As predicted, no product is seen for P1 who is homozygous for the trait (A/A); whereas, the ψ-heterozygote (G/A) shows a product. With exception of P2, all other wild-type homozygotes (G/G) show the predicted product (P3, P4, P5) and the NTC is negative. The lack of a product for P2 may reflect an error when the reaction was setup, especially noting that P2's DNA was used to make the ψ-heterozygote. In addition to 1.75× K1/TPIIB mix, other components of the Factor-V reaction included 0.8 μM forward and reverse primers, 0.2 mM dNTP (each), 3 mM MgCl₂, 1 μL of buccal cell gDNA and 4.8 units of Bst DNA Polymerase in a 15 μL reaction volume. Reaction time was 1 hr at 60° C.

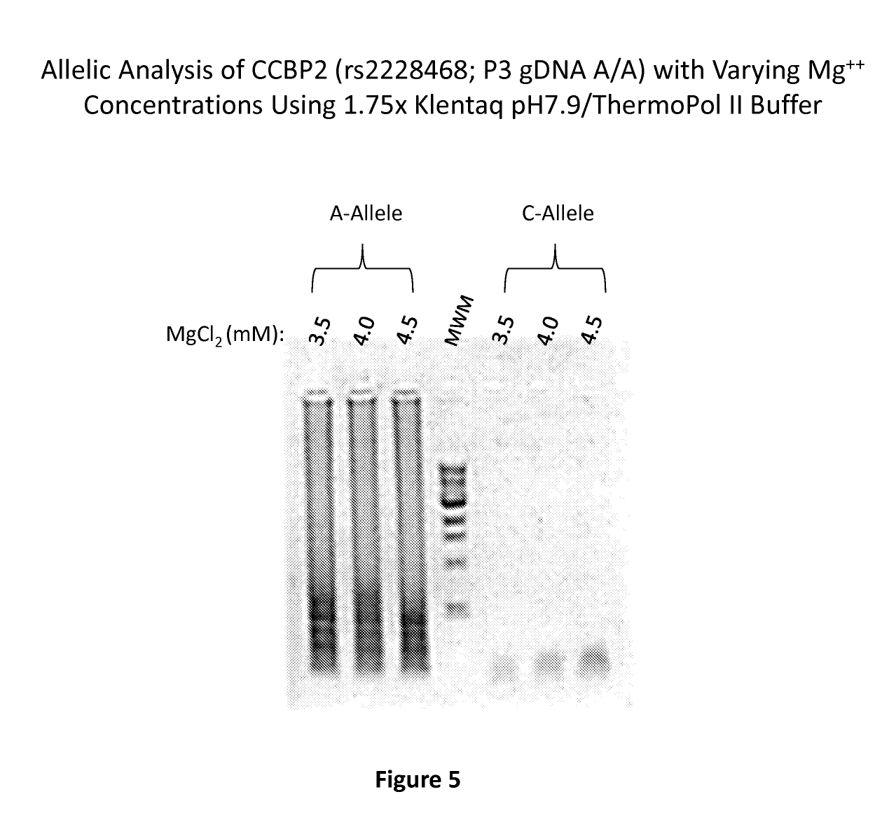

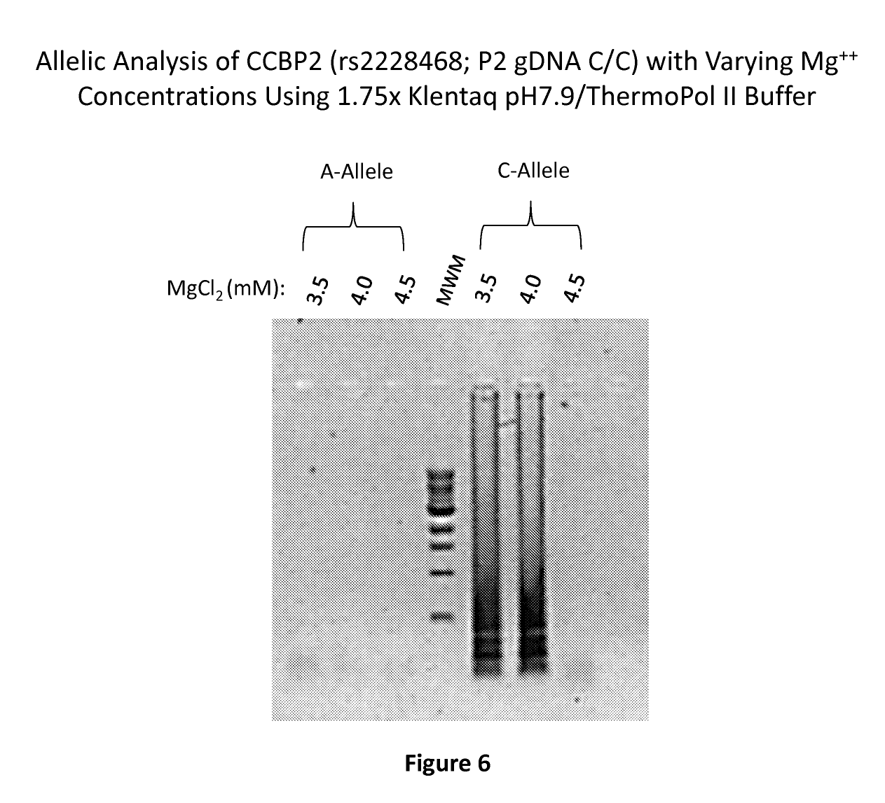

FIGS. 5 and 6 assess the SNP, rs2228468 (A or C) of the chemokine binding protein 2 (CCBP2). As determined in earlier studies, P3 is homozygous for the A-allele (A/A) and P2 is homozygous for the C-allele (C/C). Specifically, FIG. 5 shows the analysis of gDNA from P3 (A/A) with the A-allele and the C-allele amplicon (and three different MgCl₂ concentrations for each allele). As predicted, the A-allele amplicon demonstrates robust products for all Mg⁺⁺ concentrations, whereas the C-allele is negative for all. FIG. 6 shows the analysis of gDNA from P2 (C/C) for the same CCBP2 alleles. Again, as predicted, no amplification is seen with the A-allele, but the C-allele amplification is seen with 2 of the 3 MgCl₂ concentrations. Therefore, the alleles were accurately predicted for both the A- and C-alleles of CCBP2. In addition to 1.75× K1/TPIIB mix, other components of the CCBP2 reaction included 0.8 μM forward and reverse primers, 0.2 mM dNTP (each), variable MgCl₂, 1 μL of buccal cell gDNA and 4.8 units of Bst DNA Polymerase in a 15 μL reaction volume. Reaction time was 1 hr at 60° C.

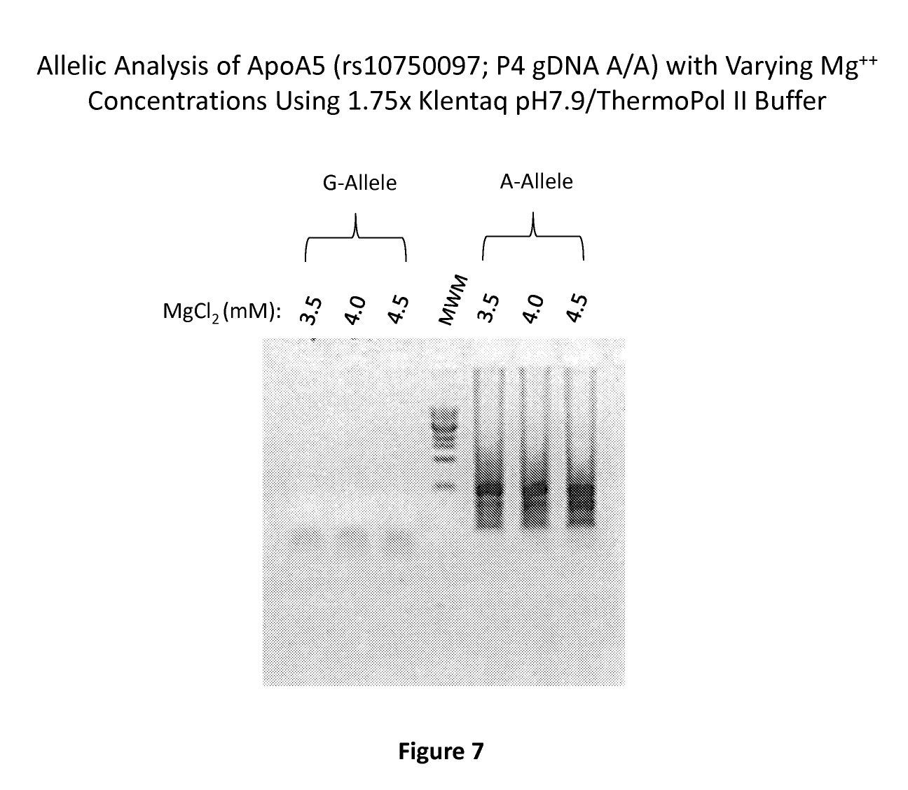

FIG. 7 assesses the SNP rs10750097 (A or G) of the apolipoprotein A-V gene (ApoA5). Here, gDNA from P4 was tested for both ApoA5 alleles (A/G) with three different MgCl₂ concentrations. Three robust reactions are seen for the A-allele only, indicating that P4 is homozygous for the A-allele (A/A). This result was later verified using RFLP-analysis of the ApoA5 gene for P4. Also, as shown later, the specificity of the G-allele reaction was verified in individuals with this SNP. In addition to 1.75× K1/TPIIB mix, other components of the ApoA5 reaction included 0.8 μM forward and reverse primers, 0.2 mM dNTP (each), variable MgCl₂, 1 μL of buccal cell gDNA and 4.8 units of Bst DNA Polymerase in a 154 reaction volume. Reaction time was 1 hr at 60° C.

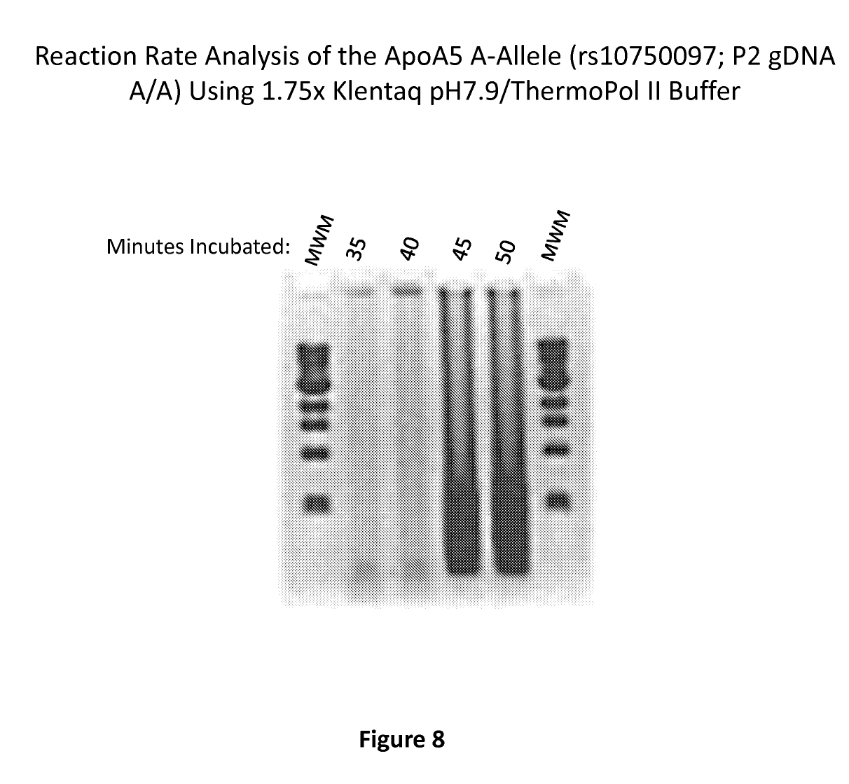

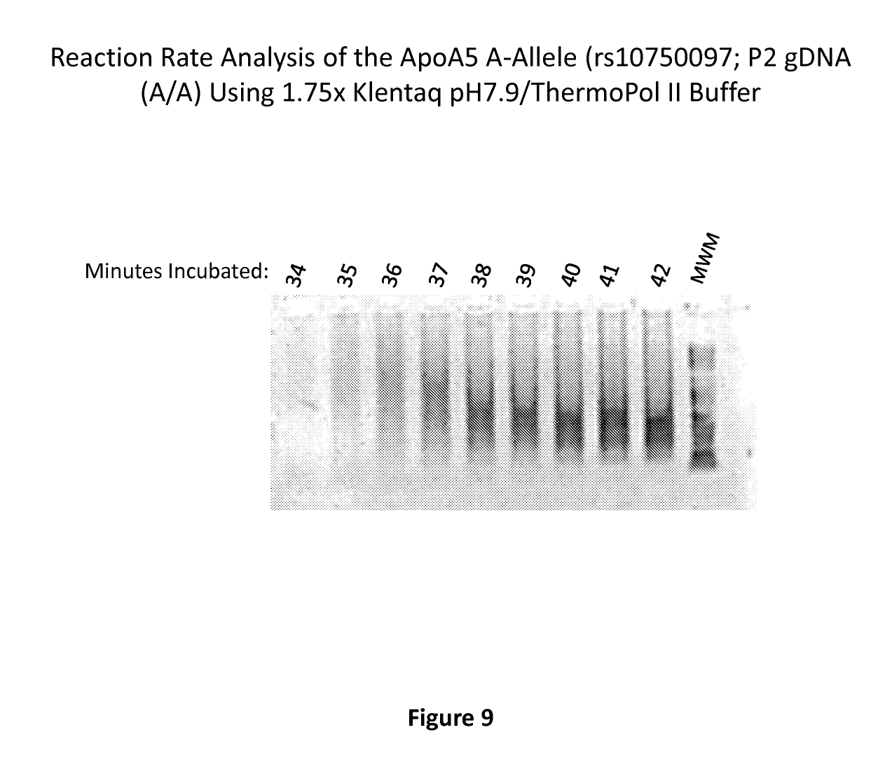

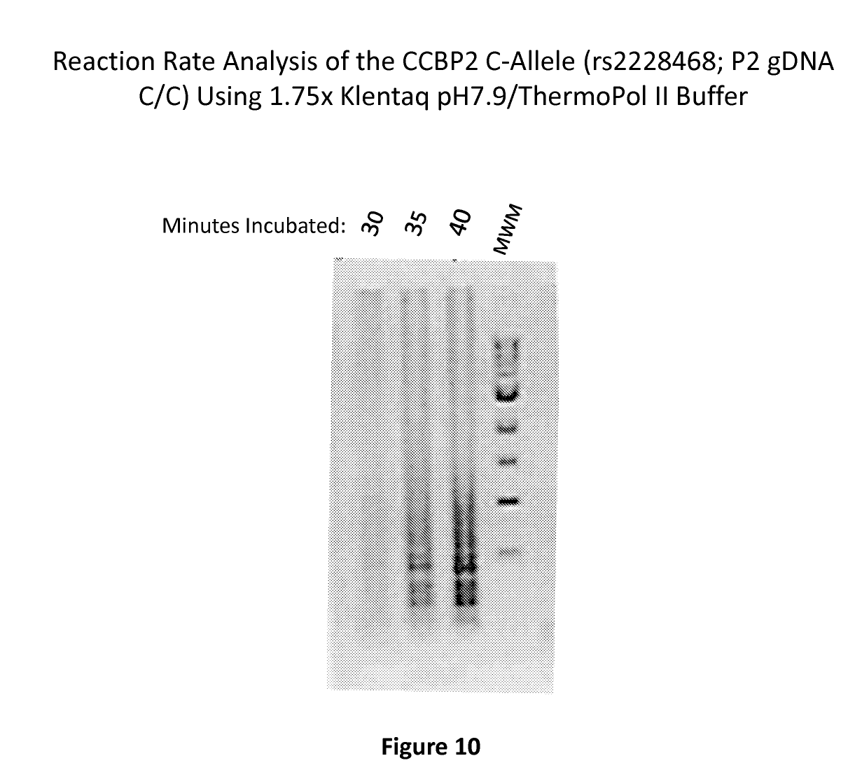

It was evident from the above results that by 60 minutes the reactions were maximized with the primer design and buffer conditions. Therefore, more data was needed concerning the actual rates of reaction. With this established, the reaction conditions were further optimized to push the system faster. FIG. 8 shows a time course analysis of the A-allele from the ApoA5 SNP, rs10750097. Using gDNA from P2, who is homozygous for the A-allele (A/A), four identical reactions were set-up from a master mix then incubated at 60° C. for the indicated times, clearly a faint product was visible by 35 minutes and the reaction appeared maxed out by 45 minutes. FIG. 9 shows an even more detailed minute-by-minute time course of the ApoA5 A-allele, also using P2 gDNA. Here, 5 μL aliquots were taken from a 50 μL master mix every one minute. FIG. 10 shows a time course study similar to FIG. 8, but with the C-allele of the CCBP2 SNP rs2228468 using gDNA from P2 who is homozygous for the C-allele (C/C). Here a product is visible at 30 minutes and appears maximized by 40 minutes. In addition to 1.75× K1/TPIIB mix, all of the above reactions included 0.8 μM forward and reverse primers, 0.2 mM dNTP (each), 3.75, 3.0, and 5.0 mM MgCl₂ (respectively), volumes vary depending on the reaction, but gDNA and Bst DNA Polymerase were added proportionally to these volumes.

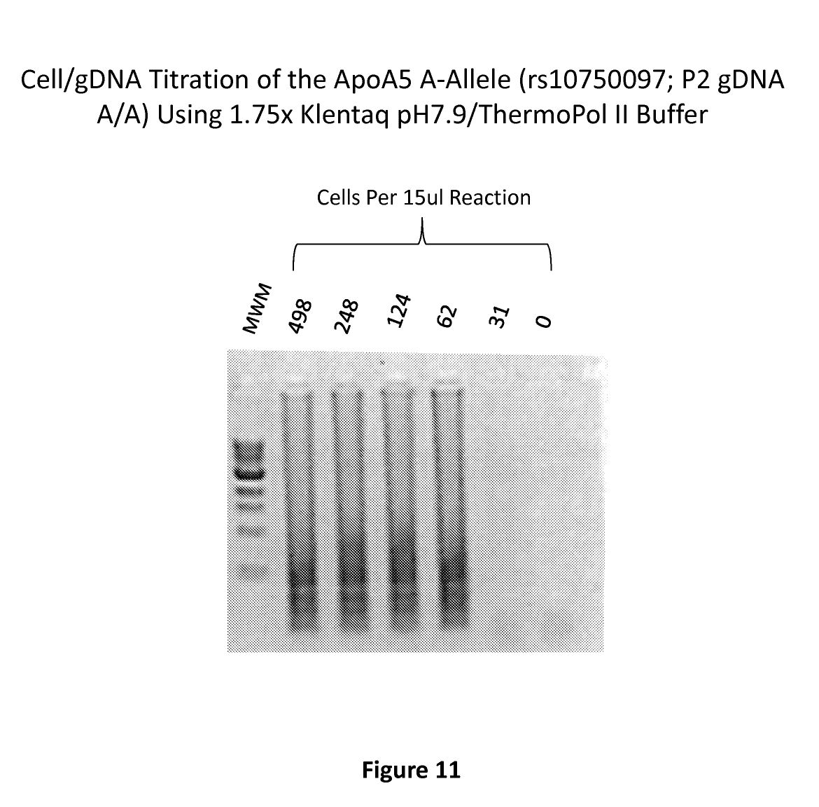

Another important aspect of the reaction was its relative sensitivity to the amount of gDNA added to the reaction. To assess this, a buccal (cheek) swab was taken from P2 with a Q-tip and the cell yield was determined by staining the cells with trypan blue and counting them on a hemocytometer. From here, a certain percentage of the cells were lysed in QuickExtract (QE) Solution (cat#QE09050, Epicentre) to give a concentration of ˜496 cells/μL. The cell lysates were then serially diluted in additional QE solution by factors of 2. FIG. 11 demonstrates the sensitivity of the ApoA5 A-allele with the cell lysate dilutions. After incubating the reactions for 60 minutes at 60° C., a strong reaction product is seen with as few as 62 cells in a 154 reaction; therefore, 120 DNA copies per reaction. It is estimated that a standard buccal cell swab, added directly to QE solution, yields an average of about 200 to 400 gene copies/μL. In addition to 1.75× K1/TPIIB mix, other components of the ApoA5 reaction included 0.8 μM forward and reverse primers, 0.2 mM dNTP (each), 3.75 mM MgCl₂, and 4.8 units of Bst DNA Polymerase in a 15 μL reaction volume.

With a better understanding of the reaction rate and sensitivity of the system, the reaction buffer was then dissected by isolating individual components and varying their concentrations over a broad range. Using 1× ThermoPol buffer as a starting point and keeping the 1.75× Klentaq1/ThermoPol II buffer mix as a point of reference, the analysis began by varying the amount of Tris-HCl added to the reactions. Noting success with the lower pH Tris-HCl in previous studies, pH 8.0 was used as a starting point. The other components of the reaction were basically kept the same as 1× ThermoPol II buffer, with 10 mM KCl and 10 mM (NH₄)₂SO₄. However, for this series of studies, the TritonX-100 was inadvertently set at 0.2% versus the standard 0.1%; this mistake was realized after the experiments had been initiated. Nevertheless, since the TritonX-100 would be subject to its own titration, and 0.2% was in line with the detergent concentrations of the 1.75× Klentaq1/ThermoPol II buffer mix, it was not changed. Also for this series of studies, for 10 μL reactions, the MgCl₂ concentration was kept at 3 mM since reaction success had been seen around this range, and the final dNTP concentrations were kept at 0.2 mM each. Forward and reverse primer concentrations were kept at 0.8 μM (each), 3.2 units of Bst DNA polymerase and 0.7 μL of buccal cell gDNA (QE solution) were added per reaction.

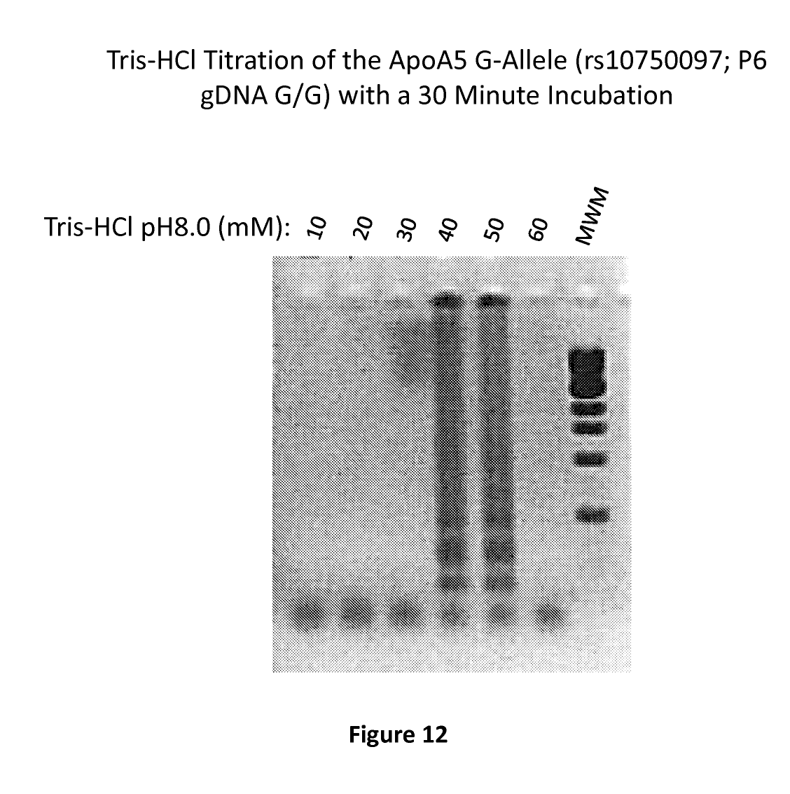

FIG. 12 shows the effect of increasing amounts of Tris-HCl pH 8.0 on the ApoA5 G-allele (rs10750097) reaction after 30 minutes incubation using P6 gDNA. Robust reaction products are seen with the 40 and 50 mM Tris-HCl pH 8.0, whereas no products are apparent with the 10, 20, 30, and 60 mM concentrations. These results suggest that optimal Tris-HCl is in the 40 to 50 mM range. Consequently, the Tris-HCl pH 8.0 concentration is optimized at 45 mM and the next focused parameter was the KCl levels. Additionally, noting the robustness of the reaction at 30 minutes, the incubation time was decreased to 25 minutes so as to be closer to the visible threshold level of the reaction (i.e., visible by gel-electrophoresis).

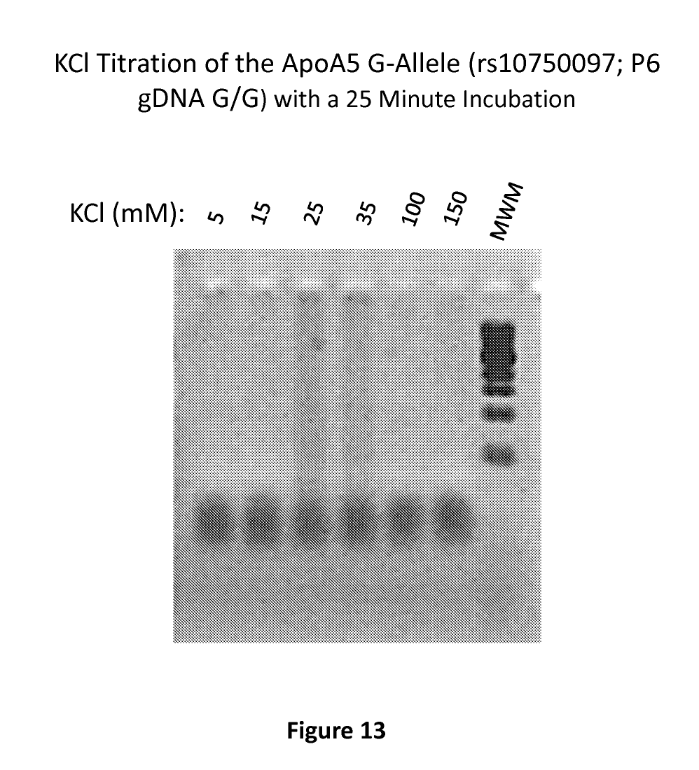

FIG. 13 shows the effect of increasing amounts of KCl on the ApoA5 G-allele (rs10750097) reaction after 25 minutes incubation using P6 gDNA (with 45 mM Tris-HCl pH 8.0, 10 mM (NH₄)₂SO₄, 0.2% TritonX-100 and 3 mM MgCl₂). Though faint, reaction products are evident with the 25 and 35 mM KCl levels, whereas little product is seen with 5, 15, 100, and 150 mM KCl. The KCl concentration was optimized at 25 mM (and Tris-HCl pH 8.0 at 45 mM), and the next focused parameter was the (NH₄)₂SO₄ levels.

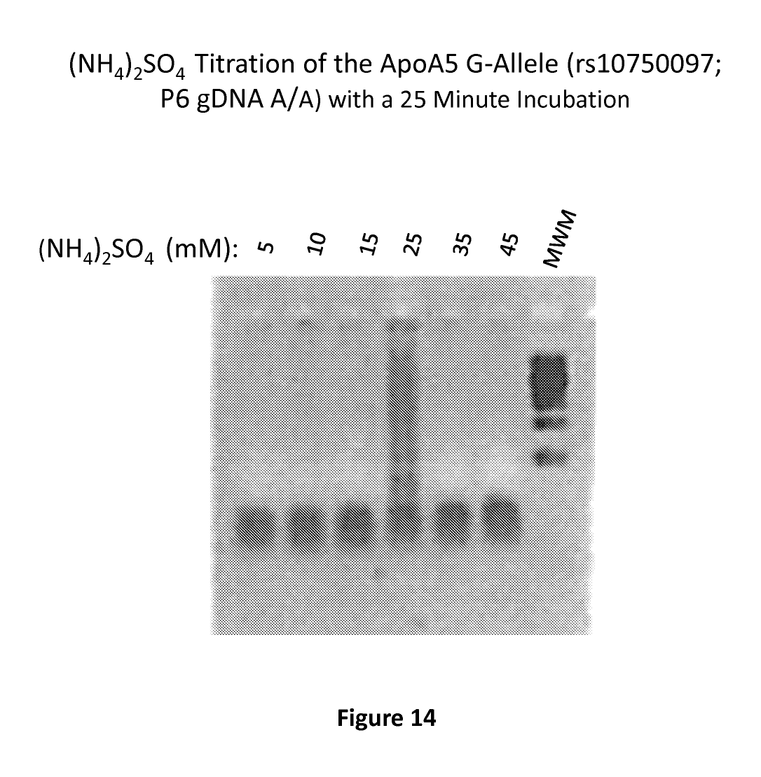

FIG. 14 shows the effect of increasing amounts of (NH₄)2SO₄ on the ApoA5 G-allele (rs10750097) reaction after 25 minutes incubation using P6 gDNA (with 45 mM Tris-HCl pH 8.0, 25 mM KCl, 0.2% TritonX-100 and 3 mM MgCl₂). A reaction product (more intense than FIG. 13) is evident with the 25 mM (NH₄)₂SO₄ level, whereas, the 5, 10, 15, 35 and 45 mM (NH₄)₂SO₄ levels show no or limited product. Next, the (NH₄)2SO₄ concentration was held at 25 mM (and Tris-HCl pH 8.0 at 45 mM and KCl at 25 mM) while increasing TritonX-100 levels.

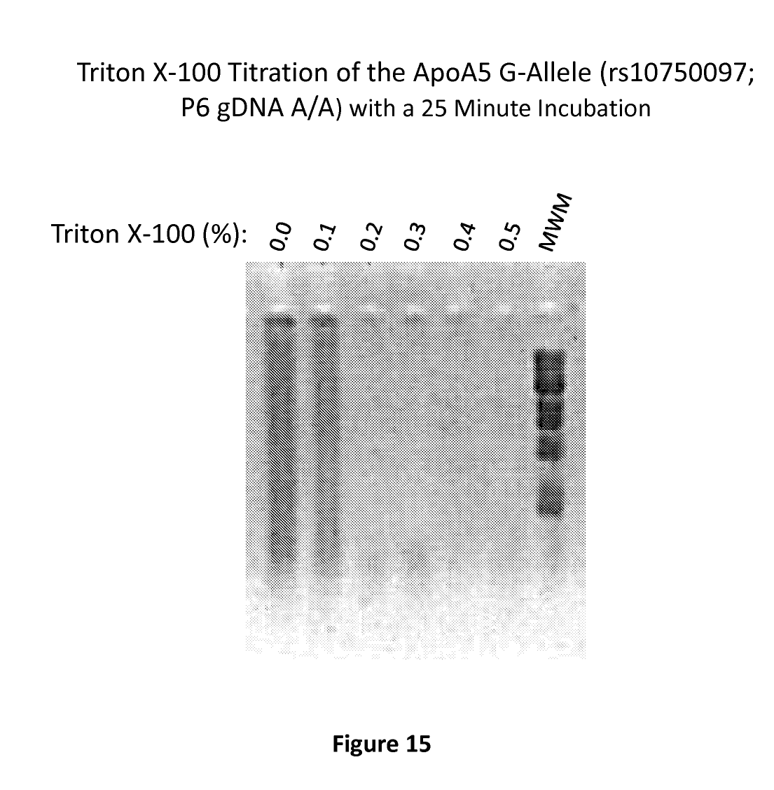

FIG. 15 shows the effect of increasing amounts of TritonX-100 on the ApoA5 G-allele (rs10750097) reaction after 25 minutes incubation using P6 gDNA (with 45 mM Tris-HCl pH 8.0, 25 mM KCl, 25 mM (NH₄)₂SO₄ and 3 mM MgCl₂). Reaction products are evident with no TritonX-100 and 0.1% TritonX-100, whereas no products are seen with 0.2%, 0.3%, 0.4%, and 0.5% TritonX-100. Why no product is seen with 0.2% TritonX-100 is not clear noting its use in the above reaction; however, it may reflect reaction to reaction variability especially at the threshold levels. Nevertheless, it is apparent that TritonX-100 has the potential to be inhibitory, and even at the standard 0.1%, it has no evident advantage over 0.0% detergent added, at least under these conditions.

Therefore the new formulation for the buffer was:

1×SNP/LAMP Buffer
45 mM Tris-HCl pH 8.0 (25° C.)
25 mM KCl
25 mM (NH₄)₂SO₄
3 mM MgCl₂

From here, minor adjustments were made to the system including raising the dNTP concentration from 0.2 mM each to 0.25 mM each, and the forward and reverse primer concentrations from 0.8 μM to 0.825 μM. Nevertheless, there seems to be quite a bit of flexibility with dNTP and primer concentrations, especially at lower levels. Additionally, as discussed below, allele specificity and reaction rates become more consistent if chemical enhancers, such as DMSO and/or betaine are added to the reactions.

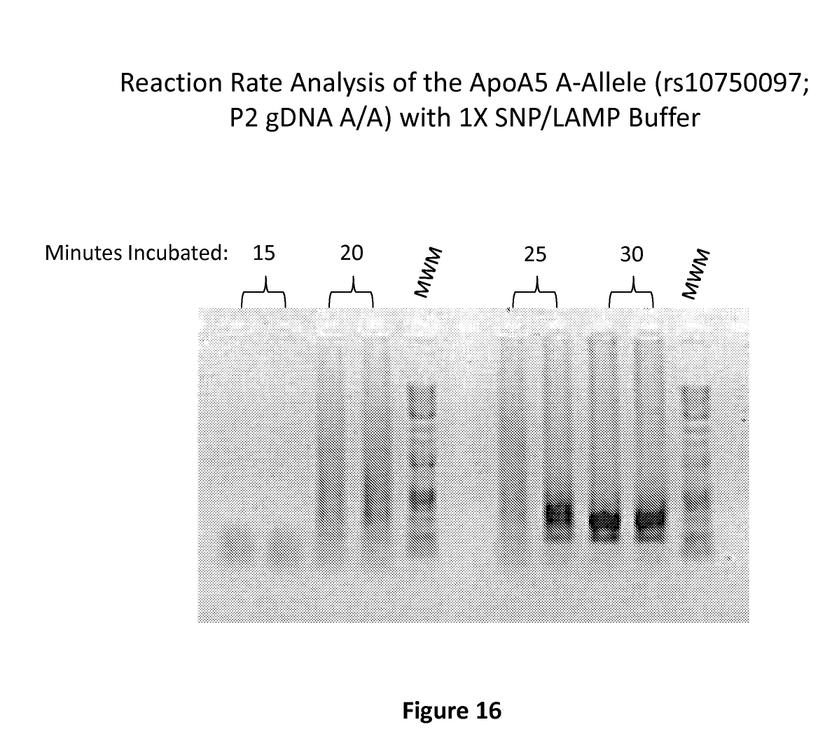

FIG. 16 shows a reaction time course of the ApoA5 A-allele with the above 1× SNP/LAMP buffer, 0.25 mM dNTPs and 0.825 μM forward and reverse primers, 0.7 μL buccal cell gDNA (QE solution.) and 3.2 units of Bst DNA polymerase in 10 μL reaction volumes. Clearly, the reactions are evident by 20 minutes and maxed out by 30 minutes. (All reactions in duplicate).

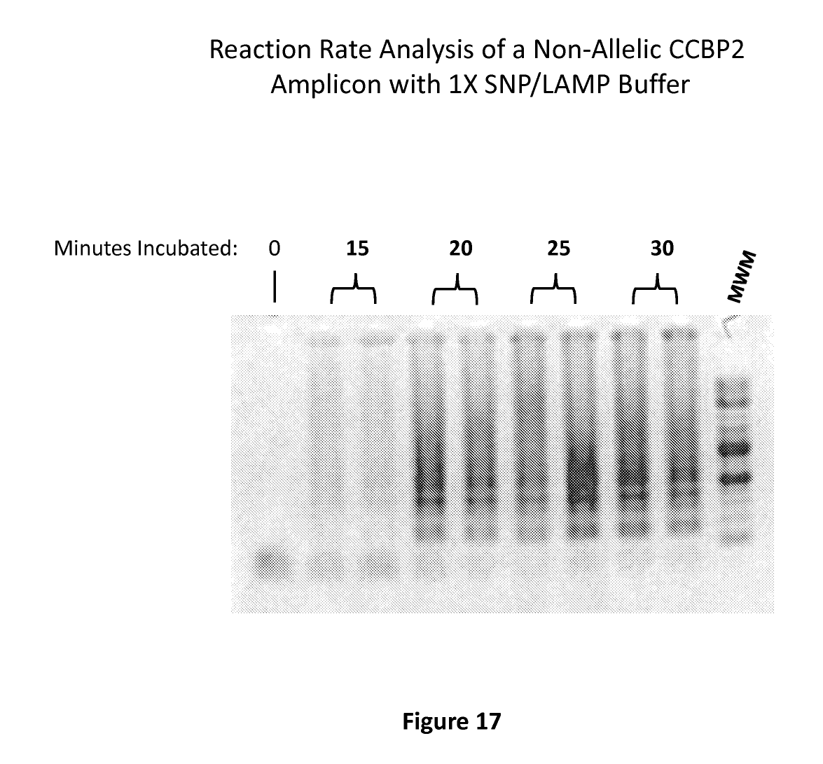

Using identical reaction conditions as FIG. 15, except with reaction volumes being 5 μL instead of 10 μL, FIG. 17 shows a reaction time course of a SNP/LAMP amplicon that flanks the CCBP2 allele described above. Here, a reaction product is visible by 15 minutes. Since this amplicon does not target a specific allele (SNP), this may allow the reaction to occur even slightly faster. (All reactions in duplicate, except for 0 minutes).

Another aspect of the buffer system is that, in some embodiments, it should be able to accurately discriminate SNPs for a given gene targeted. To address this, 1× SNP/LAMP buffer was used in a large scale reaction with the A-allele and G-allele of the ApoA5 SNP, rs10750097, where the SNP discriminating primers are 3′-SD-FIPs. Each allele was assessed in a set of five of 5 reactions including separate sets for no template controls. This was done to address the possibility of variability within a given reaction. In addition to using the 1×SNP/LAMP buffer, these reactions used 0.55 μM forward and reverse primers, 0.2 mM dNTPs (each), 0.7 μL buccal cell gDNA (QE soln.) and 3.2 units of Bst DNA Polymerase in 10 μL reaction volumes.

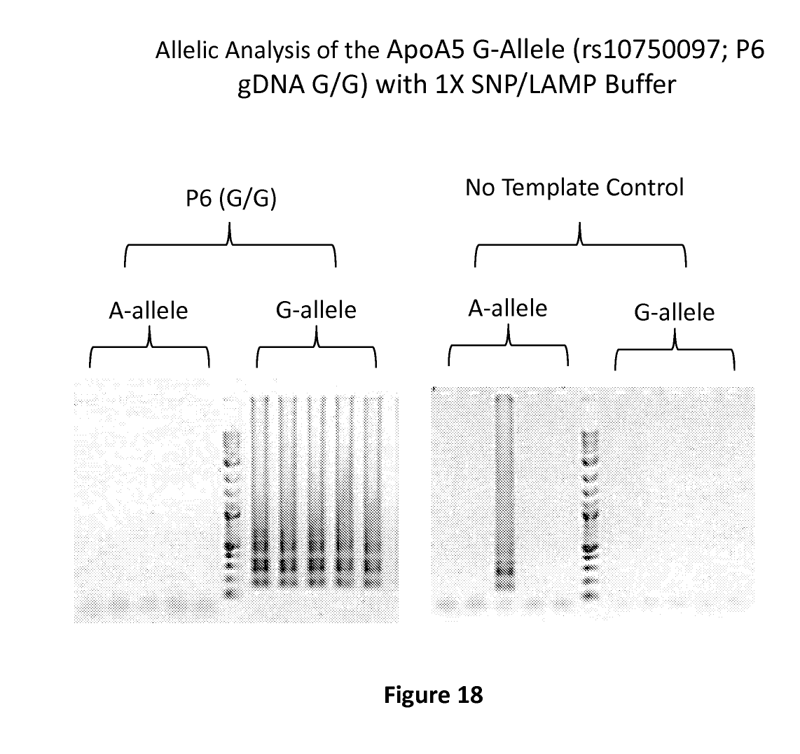

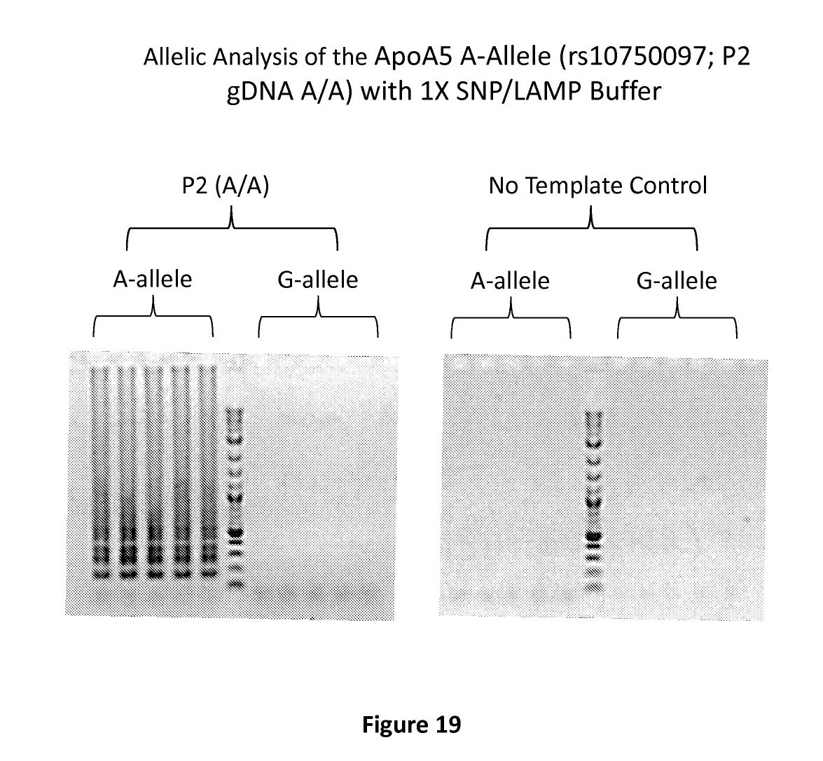

The top panel of FIG. 18 assesses the gDNA of P6, a subject who is homozygous-G (G/G) for this ApoA5 allele. As predicted, even after 40 minutes of incubation, robust products are seen for 5 of 5 of the G-allele reactions and no products, 0 of 5, are seen for the A-allele reactions. In the lower panel, which assesses the no template controls (NTCs), 1 of 5 reactions has a product for the A-allele, which would be indicative of low level contamination. The G-allele for the NTCs is negative for all 5 reactions. In FIG. 19, the reciprocal study was carried out with P2 gDNA, who is homozygous-A (A/A) for the ApoA5 allele. As predicted, the top panel shows that only the A-allele reactions have products (5 of 5) for P2 gDNA, whereas the G-allele has no products (0 of 5). Also as predicted, the lower panel shows that neither allele amplified a product in the NTC sets.

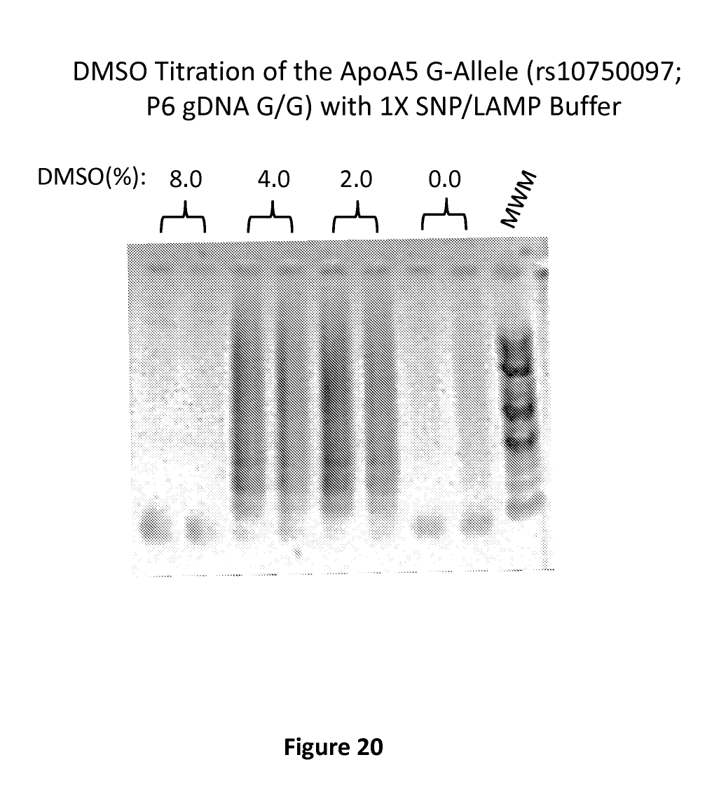

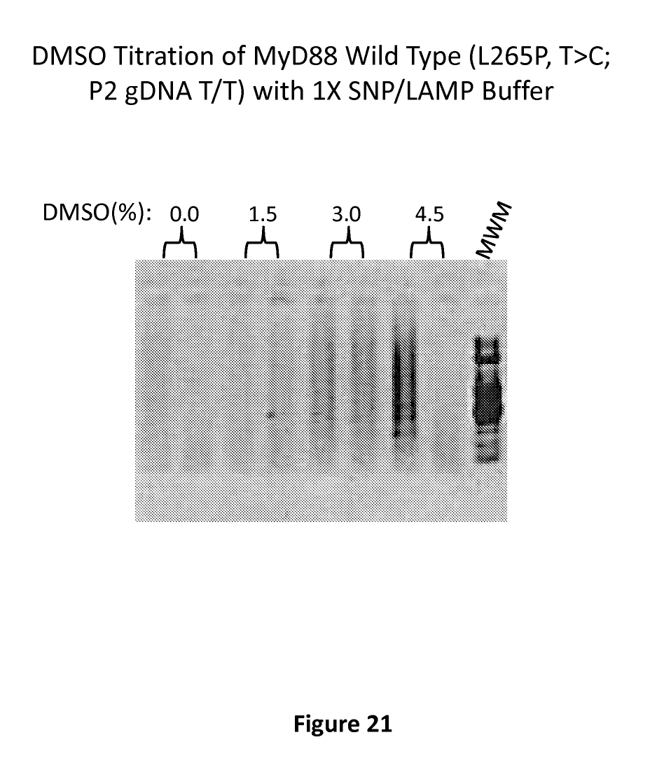

As mentioned earlier, the addition of enhancers to DNA polymerase reactions, such as betaine, DMSO, formamide, and bovine serum albumin (BSA), can have a significant influence on a reaction's specificity and efficiency, and this is especially evident with G/C-rich targets or targets with unusual secondary structures. Most studies involving isothermic reactions, including LAMP, use betaine as an enhancer, though DMSO and leucine have been proposed. FIG. 20 shows how the addition of DMSO can enhance the rate of a SNP/LAMP reaction. This reaction also targets the ApoA5 G-allele of rs10750097 with P6 gDNA, but uses 5′ SD-FIP and 5′ SD-BIP primers. Clearly, at a threshold reaction time (20 min), 2% and 4% DMSO enhance the rate of reaction as compared to 0% DMSO; 8% DMSO is likely to be inhibitory (all reactions in duplicate). FIG. 21 also shows the beneficial effects of DMSO at a threshold reaction time (20 min; all reactions in duplicate). This reaction targets the wild-type nucleotide “T” of the MyD88 gene which is frequently mutated to a “C” in B cell lymphomas; here too, DMSO concentrations around 3% seem optimal. In addition to using the 1×SNP/LAMP buffer, these reactions used 0.825 μM forward and reverse primers, 0.25 mM dNTPs (each), 0.35 μL, buccal cell gDNA (QE soln.) and 1.6 units of Bst DNA Polymerase in 5 μL reaction volumes.

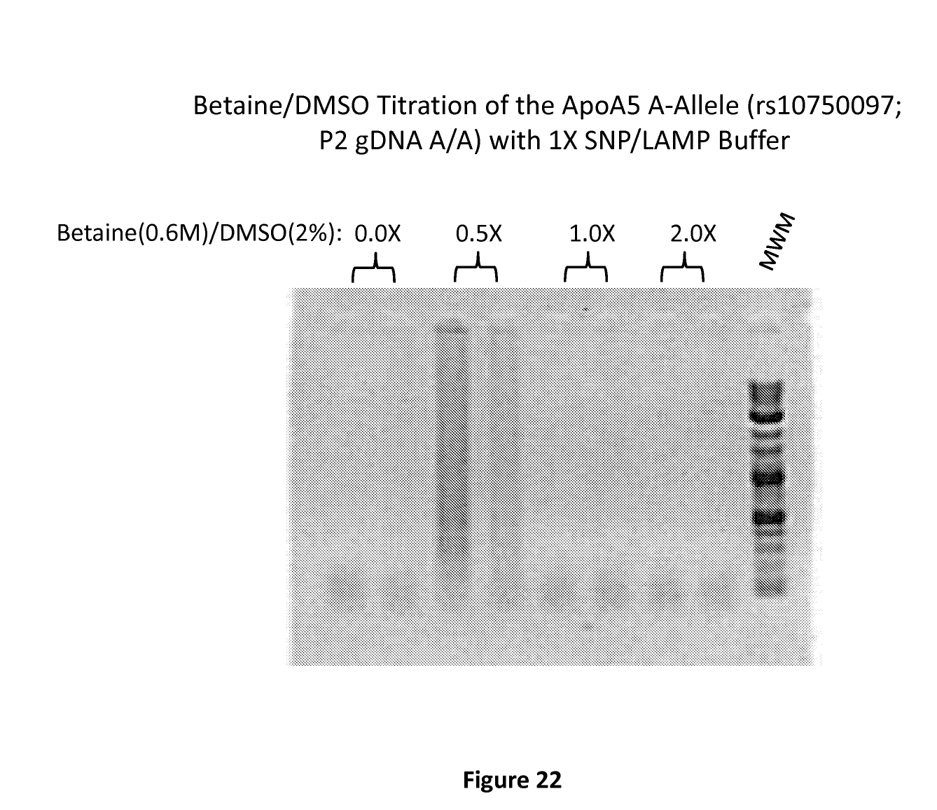

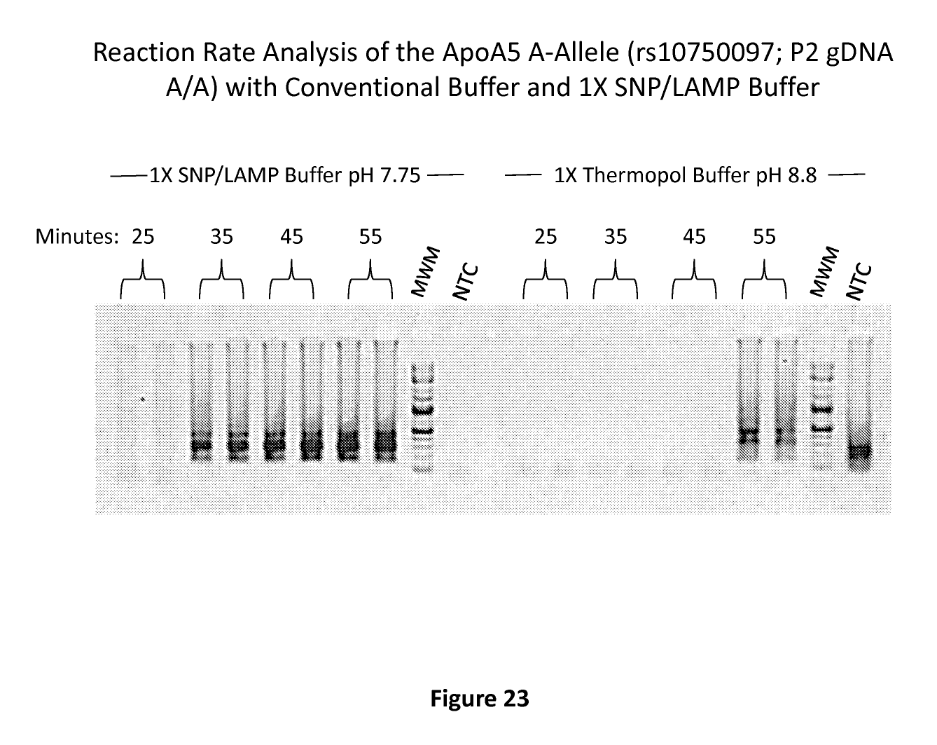

Noting the reported effectiveness of betaine in LAMP reactions, a combination of betaine and DMSO was used in the reactions where 0.6M betaine and 2% DMSO equal a 1× solution. FIG. 22 shows the titration of the enhancer with the ApoA5 A-allele (same amplicon other than FIG. 20) with P2 gDNA. Here the optimal betaine/DMSO enhancer concentration is around 0.5×, whereas 0.0×, 1.0× and 2.0× have no product at 25 minutes, which is consistent with the explanation that the 1.0× and 2.0× concentrations are inhibitory; all reactions are in duplicate. When working with a new amplicon, titrating the betaine/DMSO is a good starting point. Of note, in this reaction (as well as FIG. 23), the pH of the 45 mM Tris-HCl was 7.75. A pH lower than 8.0 was used to determine if allelic specificity and reaction time could be tweaked even further. A pH of 7.75 seems to be at the lower end of the optimal range for some amplicons, and may slow the reaction somewhat; however, SNP specificity appears improved. In addition to using the 1×SNP/LAMP buffer and DMSO, these reactions used 0.825 μM forward and reverse primers, 0.25 mM dNTPs (each), 0.35 μL, buccal cell gDNA (QE soln.) and 1.6 units of Bst DNA Polymerase in 5 μL reaction volumes.

FIG. 23 shows a time course comparison of reactions in the 1×SNP/LAMP buffer (pH 7.75) vs. NEB's standard ThermoPol II buffer (pH 8.8) using the ApoA5 A-allele amplicon with P2 gDNA. In 5 μL, both reactions have 3 mM MgCl₂, 0.25 mM dNTPs (each), 0.825 μM forward and reverse primers, 0.5× betaine/DMSO,1.6 units of Bst DNA polymerase and 0.7 μL gDNA (QE solution). Importantly, reactions are seen in the 1×SNP/LAMP reaction buffer by 25 minutes, whereas no product is seen with 1× ThermoPol buffer until 55 minutes. Additionally, as predicted, there is no product in the no template control (NTC) at 55 minutes; however, the NTC for the ThermoPol II buffer appears to have an intense non-specific product at 55 minutes; this would suggest that the 1×SNP/LAMP buffer conditions not only provide enhanced reaction rate but also offer higher specificity (all reactions in duplicate).

### Example 2

### Rapid, Accurate, SNP-Detecting LAMP Reaction

Buccal Cell gDNA Extraction

A small flocked applicator tip (Dentsply) is used to take a cheek/buccal swab. This is done by rotating the tip on the inner cheek with moderate pressure in an area of ˜4 cm². When using the applicator tip, on average about 70,000 cells are collected. The cell containing tip then goes into a one-step, extraction solution (such as QuickExtract Solution from Epicentre, cat#QE0905T). 100 μL of extraction buffer is used (ultimately, ˜700 cells/μL); the tip is submerged in a tube containing extraction buffer while rotating and pressing it against the side of the tube until the solution takes on an opaque color. The cell suspension is heated for 2-3 minutes at 65° C., then 1-2 minutes at 98° C. to prepare it for SNP/LAMP reaction.

### LAMP Initiation Using Only FIP and BIP Primers (No Outer Primers)

As depicted in FIG. 1B, via dynamic equilibrium, at elevated temperatures (60° C.), a FIP primer will strand invade its complementary target sequence on gDNA. (Of note, the equivalent event can occur on the opposite strand with BIP, but for the sake of simplicity, only one strand will be focused upon). The invading FIP primer will then be extended by Bst DNA polymerase which has strong strand displacement activity. For the reaction to proceed, a secondary FIP primer has to strand invade the position of the primary FIP. With extension of the 2° FIP, it will displace the initial extension product (S3 strand).

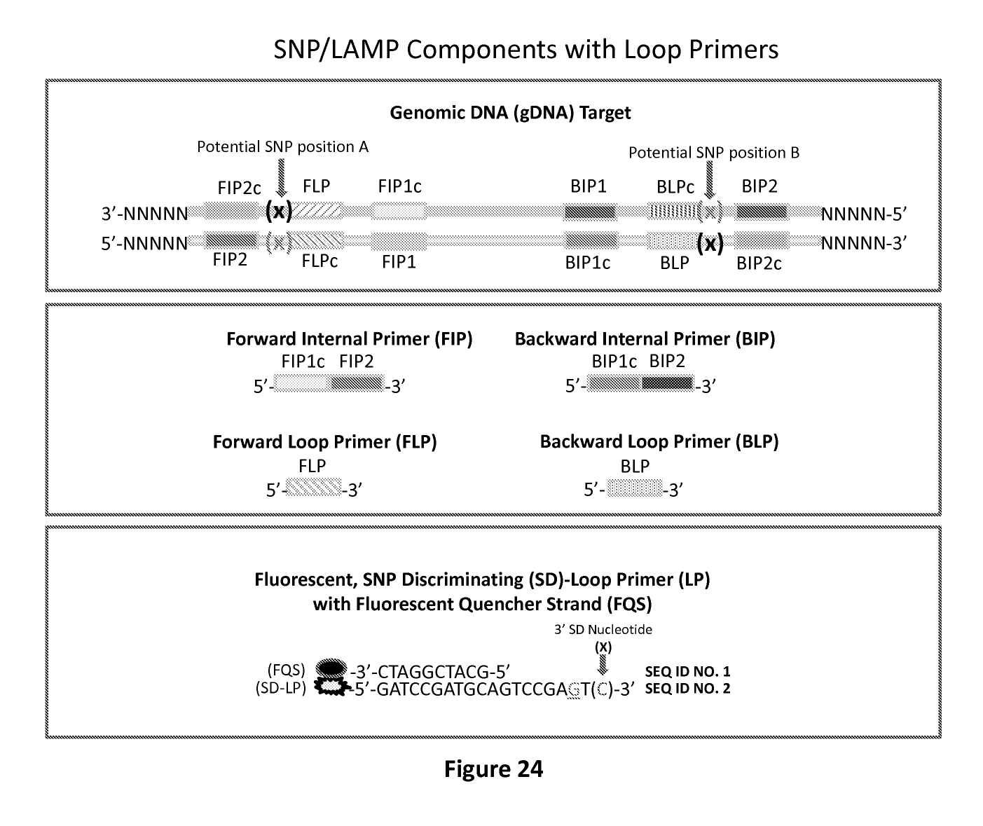

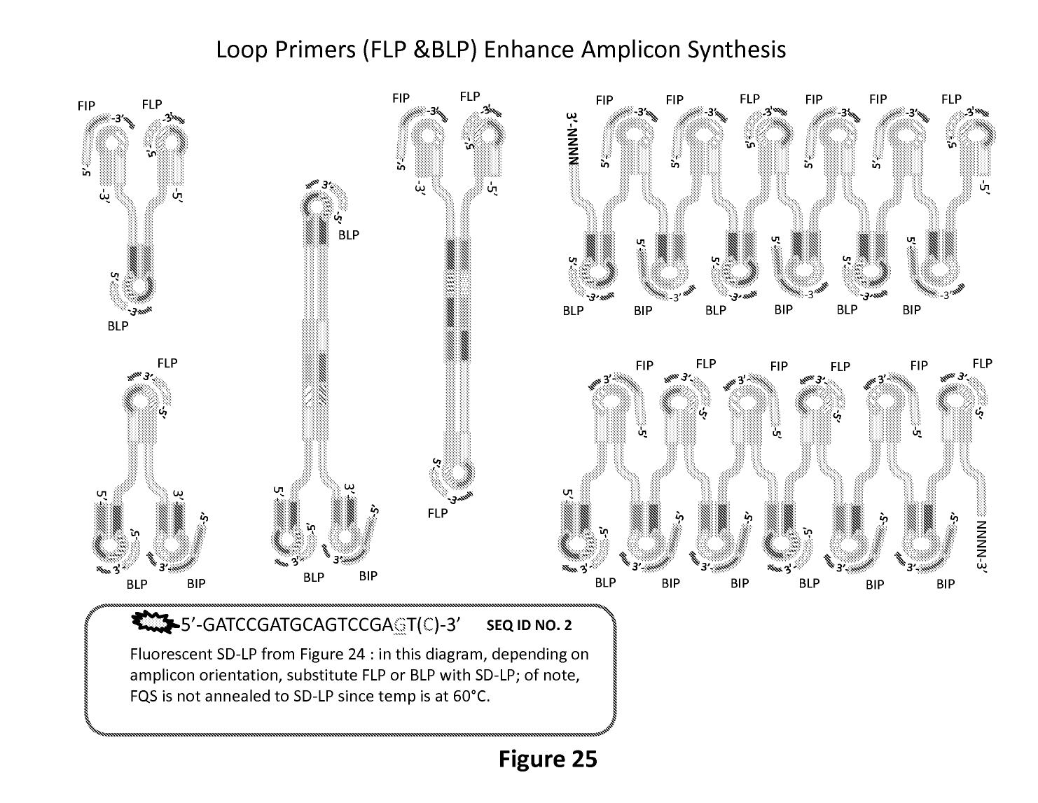

As depicted in FIG. 1C, a BIP primer will now target its complementary sequence on the displaced extension product (S3 strand), and it will be extended by Bst DNA polymerase. As with the FIP primer, a secondary BIP primer would have to invade the position of the primary BIP. Again, with extension of the 2° BIP, it will displace the initial extension product (S4 strand). The displaced strand, with its dumbbell-like structure, now represents the established amplicon. It can enter into the cyclic amplification stage (FIG. 1D). As depicted in FIGS. 24 and 25, accelerated LAMP amplification may be achieved using loop primers. Loop primers are complementary to the single stranded loop regions not targeted by FIP or BIP primers. They provide additional starting sites for DNA synthesis and accelerate the amplification, thereby further reducing the reaction time. FIG. 25 also depicts how secondary LAMP products, similar to those seen in FIGS. 1E and 1F, are targeted by loop primers, are much larger strand displaced target sequences.

SNP/LAMP Reaction Mixture with SD-LPs

For a 20 μL reaction, 1.5 to 3.0 μL of the gDNA extract is added to a reaction mixture. The reaction mixture consists of: 1× buffer system+/−a DMSO/betaine mixture as an enhancer (1× enhancer equals 2% DMSO and 0.6M betaine); the forward inner primer (FIP) and the backward inner primer (BIP) (0.825 μM each); SNP-discriminating, loop primer (SD-LP can be either a SD-BLP or SD-FLP) complex (0.2 μM); and optionally, depending on amplicon orientation, a back loop primer (BLP) or a forward loop primer (FLP) (0.2 μM) to further enhance the reaction rate. Importantly, the reaction does not use outer primers. Based on Tm, the design of the FIP, BIP, FLP, and BLP primers vary; reactions are run at 60° C. based on these Tm formulations. The reaction is run for 15-25 minutes at 60° C. and then fluorescence is visualized with a Dark Reader (Clare Chemical Research), or LED, or laser light source with the appropriate wavelength and filter.

SNP/LAMP Primer Design

As depicted in FIG. 24, the 5′ end of the SD-FLP primer is modified with a fluorescent dye, such as JOE. To quench the JOE fluorescence, a complementary strand of DNA with a 3′ quencher, i.e., BHQ1, binds the 5′-half of the LP (hereinafter “fluorescence quencher strand” or “FQS”). The FQS is designed to have a Tm of ˜40° C. Therefore, at a reaction temperature of 60° C., the FQS strand is not associated with the fluorescent SD-LP primer (FIG. 25). Consequently, the fluorescent SD-LP can bind the looped SNP region without interference from the FQS. If the SNP is present, the fluorescent SD-LP will be extended and incorporated into the reaction product and this will also accelerate the overall reaction. With termination of the reaction (15 to 25 min) and cooling temps, the FQS will rapidly reassociate with unincorporated fluorescent SD-LP, therefore quenching it. Conversely, incorporated fluorescent SD-LP (i.e., a positive SNP reaction) cannot be quenched by the FQS, hence it continues to fluoresce. In FIG. 24 and FIG. 25, (C)=the 3′ SNP discriminating nucleotide and G=an intentionally mismatched nucleotide that further enhances SD-LP's SNP specificity.

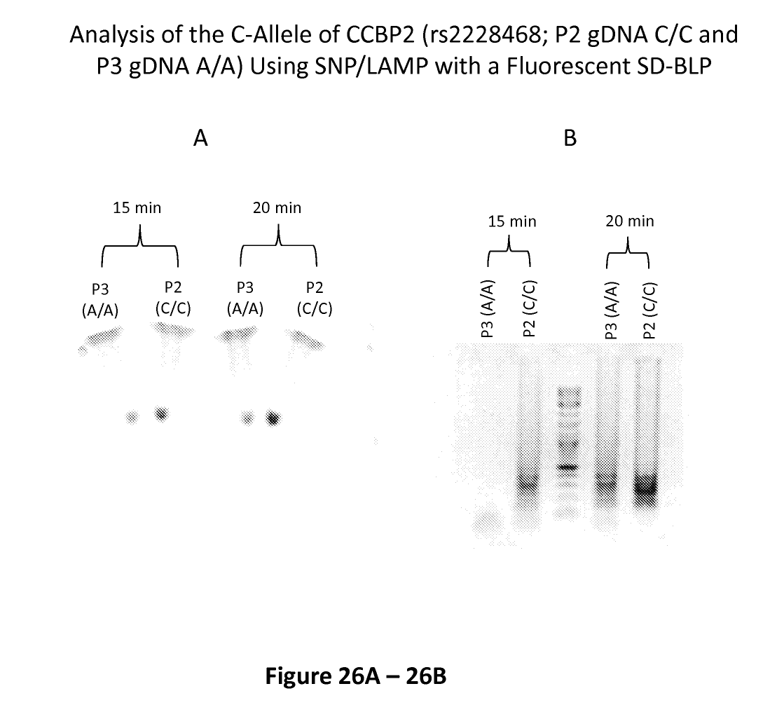

FIG. 26A shows an example of SNP/LAMP with a fluorescent SD-BLP targeting the C-allele of the CCBP2 SNP, rs2228468. DNA from P2, who is homozygous C/C for rs2228468, is demonstrating a fluorescent signal under the Dark Reader (inverted image) after 15 and 20 minutes at 60° C. Conversely, gDNA from P3, who is homozygous A/A, shows only background fluorescence for the same time points. FIG. 26B represents the same reactions as 26A, but they are now resolved on a 1% agarose gel (w/EtBr). This result demonstrates that the SD-BLP enhanced the reaction for P2 gDNA (C/C), but not the reaction for P3 gDNA (compare at 15 minutes).

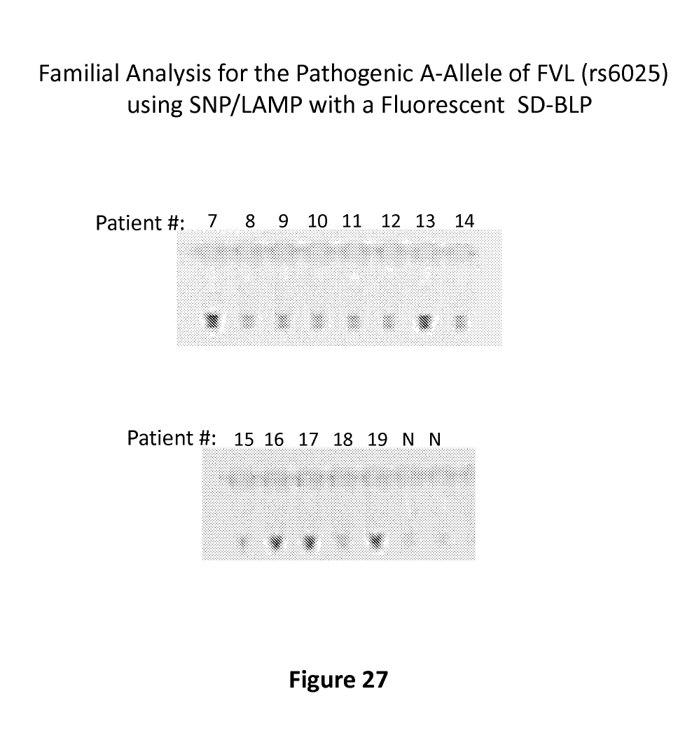

Another example of fluorescent SD-BLP is shown in FIG. 27, which depicts the screening of gDNA from family members (with a known family history) for the pathogenic A-allele of FVL (rs6025) after 25 minutes at 60° C. This result demonstrates that P7, P13, P16, P17, and P19 are positive for the trait, whereas the others are negative (the N's in this figure represent no template controls). This aligns with earlier RFLP analysis showing that P7, P13, P16, P17, and P19 are, in fact, heterozygous (G/A) for FVL (rs6025).

### Example 3

### Miscopy or Misamplification of SD-FIP and/or SD-BIP Primers During the LAMP Reaction

SNP/LAMP has the potential to be a rapid, inexpensive molecular diagnostic tool with a broad range of applications. This includes detecting subtle genetic variations such as SNPs or point mutations in a point-of-care, or point-of-use environment. Until recently, the vast majority of research on LAMP has used forward inner primers (FIP) and/or backward inner primers (BIP) to detect (or discriminate) the allelic differences of specific targets on gDNA. That is, the 3′ or 5′ nucleotide of the FIP or BIP primer is designed to match or mismatch the allelic differences of a defined SNP, or mutation, of a known gene (see FIG. 1A, lower panel). Therefore, if the 3′ or 5′ nucleotide of the FIP or BIP is complementary to the nucleotide of the targeted DNA region, the primer is extended by Bst DNA polymerase (large fragment) and the LAMP reaction is initiated. This results in the exponential amplification of a product that can be easily visualized within 20 to 25 minutes (i.e., gel electrophoresis or molecular dyes). Conversely, if the 3′ or 5′ nucleotide is a mismatch, no reaction occurs, hence, no detectable product.

The use of FIP or BIP primers as allelic discriminators is very intriguing, noting the ease of design and how robust and accurate they can be in the SNP/LAMP reaction. However, as is described herein, the potential for miscopy (undesired primer extension) from the mismatched 3′ or 5′ nucleotides is a possibility; and as has been discussed, once a spurious reaction is initiated from FIP or BIP, via miscopy, it will be amplified into a false positive.

The underlying mechanisms of miscopy (also referred to in the literature as misamplification) have proven very difficult to discern, especially noting its arbitrary nature. Miscopy, however, is not rare and others have gone to considerable measures to suppress this phenomenon. The rate of miscopy can vary widely from nonoccurrence, even in large studies, to levels greater than 50%. Nevertheless, when miscopy does occur, several variables may apply (either by themselves or in combination). These variables include the overall length of the incubation time, the gene being targeted, the quality of the primers (over time) and gDNA, contaminants in buffers or DNA preps, and perhaps the age or condition of the polymerase. Regardless, the threat of miscopy requires reconsideration of how to target SNPs using LAMP, and this led to the development of fluorescent, SNP-discriminating loop primers (SD-LPs). Since fluorescent SD-LPs are loop primers, they can only enhance the LAMP reaction but by themselves can't drive the reaction (FIG. 26); consequently, if a miscopy event occurs from an allele-specific loop primer it is not likely to amplify into a false-positive.

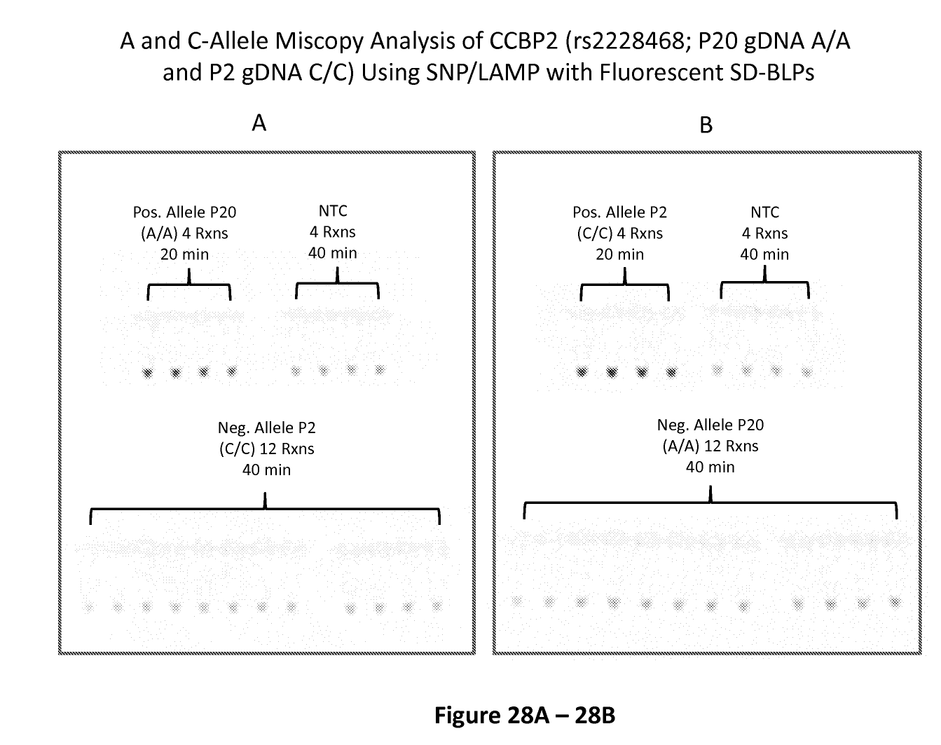

FIGS. 28A-28B depicts miscopy analysis of the A-allele and C-allele of CCBP2, rs2228468, using SNP/LAMP fluorescent SD-BLPs. FIG. 28A, which assesses the A-allele, shows that gDNA from P20, a patient homozygous A/A, is positive (fluorescent) for 4 out of 4 reactions after 20 minutes at 60° C.; whereas the 4 no template controls (NTC) and the 12 negative allelic controls, with P2 gDNA (C/C), are negative (background fluorescent) after 40 minutes at 60° C. FIG. 28B represents the reciprocal, C-allele, study of 28A with identical reaction numbers and incubation times. Here too, the predicted fluorescence pattern is demonstrated for the positive controls (P2 gDNA) and the negative controls (P20 gDNA and NTC). Fluorescence for all reactions was visualized with a Dark Reader and inverted images are shown.

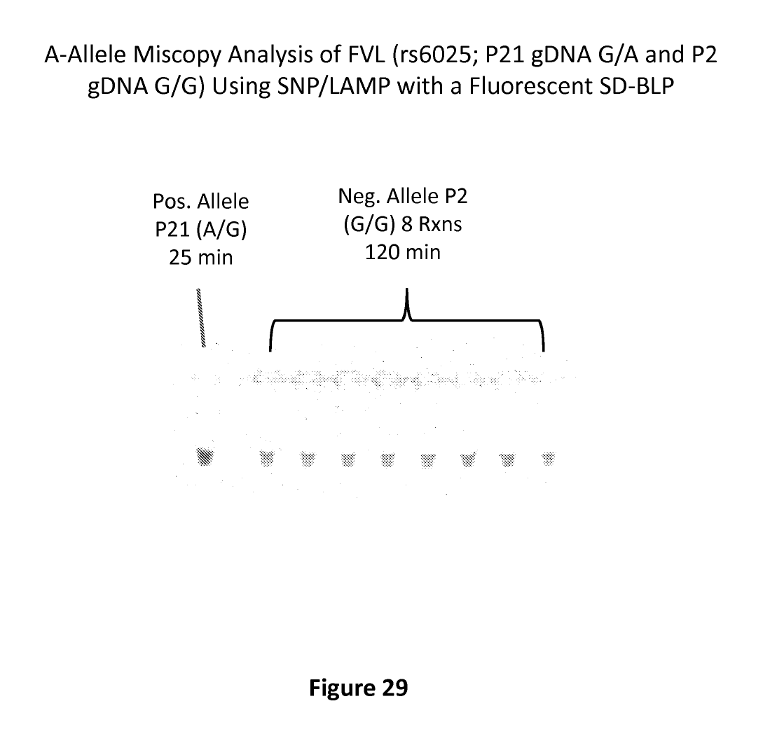

FIG. 29 depicts miscopy analysis of the pathogenic A-allele of FVL (rs6025) using SNP/LAMP, fluorescent SD-BLP. Here, gDNA from P21, a patient heterozygous (A/G) for the A-allele, shows a fluorescent signal after 25 minutes at 60° C. Conversely, 8 negative allelic controls with gDNA from P2 (G/G) show only background fluorescence even after 120 minutes at 60° C., 4× the incubation time relative to the control.

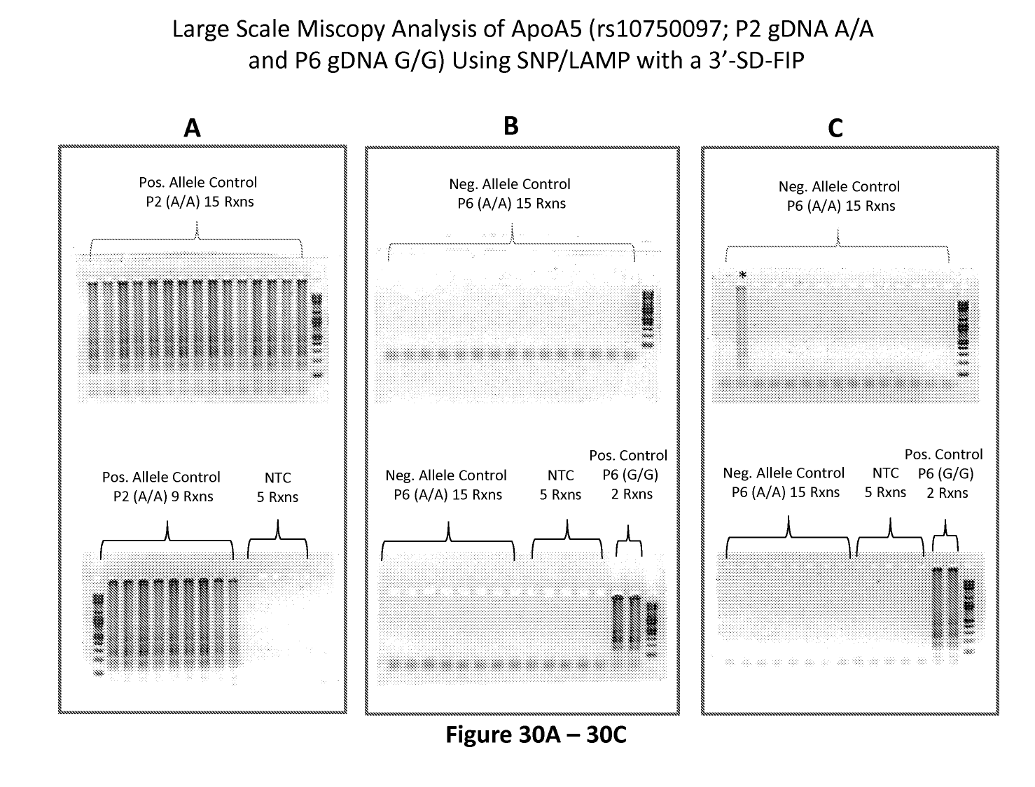

FIGS. 30A-30C depicts a large scale allelic control study to assess the frequency of miscopy amplification relative to allele-specific amplification using FIP and BIP primers as SNP discriminators (in the particular study described here, FIP is the SNP discriminator). FIG. 30A shows the positive allele assessment for the A-allele (rs10750097) of the ApoA5 gene. All 24 reactions that contain P2 gDNA (A/A) are positive. Furthermore, the no template controls (NTC) show no amplification. For FIG. 30B, the A-allele of ApoA5 is also being assessed but uses P6 gDNA (G/G) as a negative allelic control. As predicted, none of these G/G samples amplified. Quality of the G/G gDNA is also shown on the same gel by using primers targeting the ApoA5 G-allele which readily amplify; again, no products are in the NTC wells. FIG. 30C shows the same study as FIG. 30B, but a single miscopy event has occurred (indicated by the asterisk*). These reactions represent a very good example of SNP/LAMP'S potential (using FIP or BIP as SNP discriminators) when the system is behaving ideally. Reactions were run at 60° C. for 20 minutes and unmodified FLP and BLP primers are present.

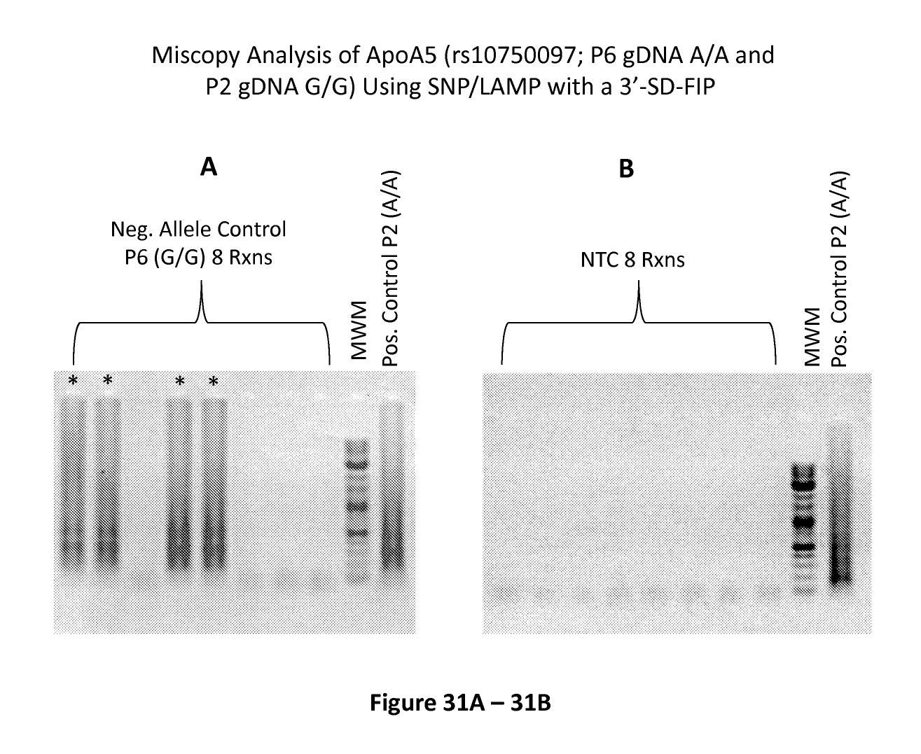

FIGS. 31A-31B depicts miscopy phenomena using the same ApoA5 SNP primers as FIGS. 30A-30C. FIG. 31A shows that 50% of the negative allelic controls (G/G) are positive for the A-allele (indicated by the asterisks*). Whereas, in FIG. 31B, the NTCs from the same series of studies show no amplification; this basically eliminates the possibility that the unpredicted reactions shown in FIG. 31A are the result of amplicon contamination. A valid explanation for this result is that Bst DNA polymerase (large fragment) extends (or miscopies) the mismatched 3′nucleotide of FIP as it anneals to its target on gDNA. In this particular study the NTCs and the negative allele controls were incubated for 40 minutes, whereas the positive controls were incubated for 20 minutes. Therefore, part of the miscopy seen here may reflect the incubation time, but noting the intensity of these miscopy reactions, they were initiated many minutes earlier. Reactions were run at 60° C. and included unmodified FLP and BLP primers.

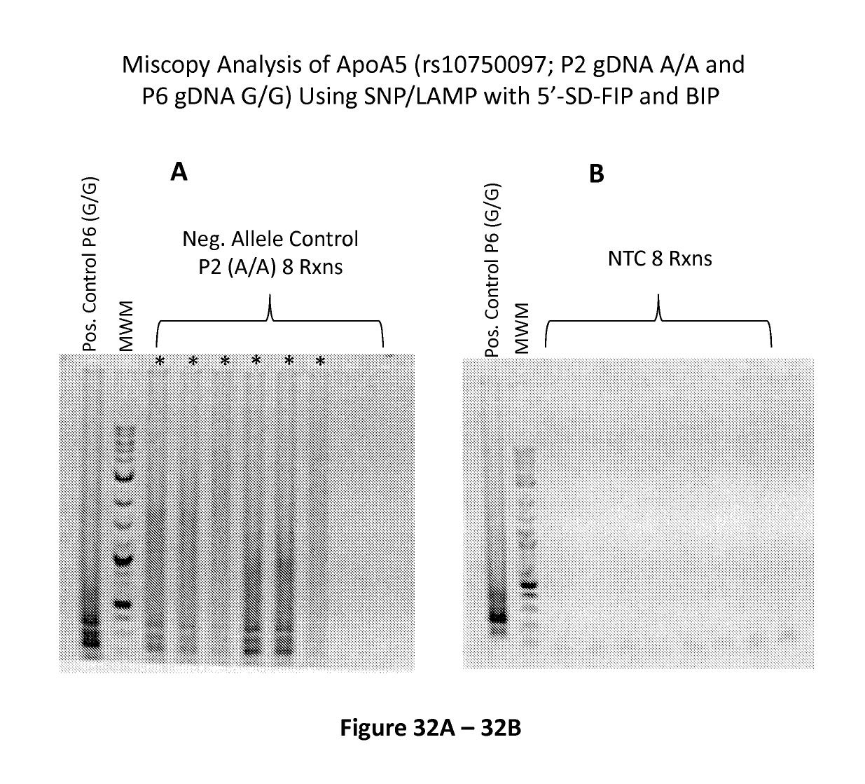

FIGS. 32A-32B depicts miscopy phenomena, however, in this circumstance the negative allelic controls and the NTCs were incubated for the same amount of time as the positive control reactions (20 minutes). These reactions were also targeting the ApoA5 SNP (rs10750097), but the FIP and BIP primers used 5′SNP-discriminating nucleotides, and homozygous A/A gDNA was used as the negative allelic control. As indicated in FIG. 32A, by the asterisks*, 6 of 8 of the A/A controls show miscopy products after 20 minutes, whereas, none of the NTCs, FIG. 32B, show products. Therefore, as with FIGS. 31A-31B, the undesired reactions are interpreted to represent miscopy. Reactions were run at 60° C. and included unmodified FLP and BLP primers.

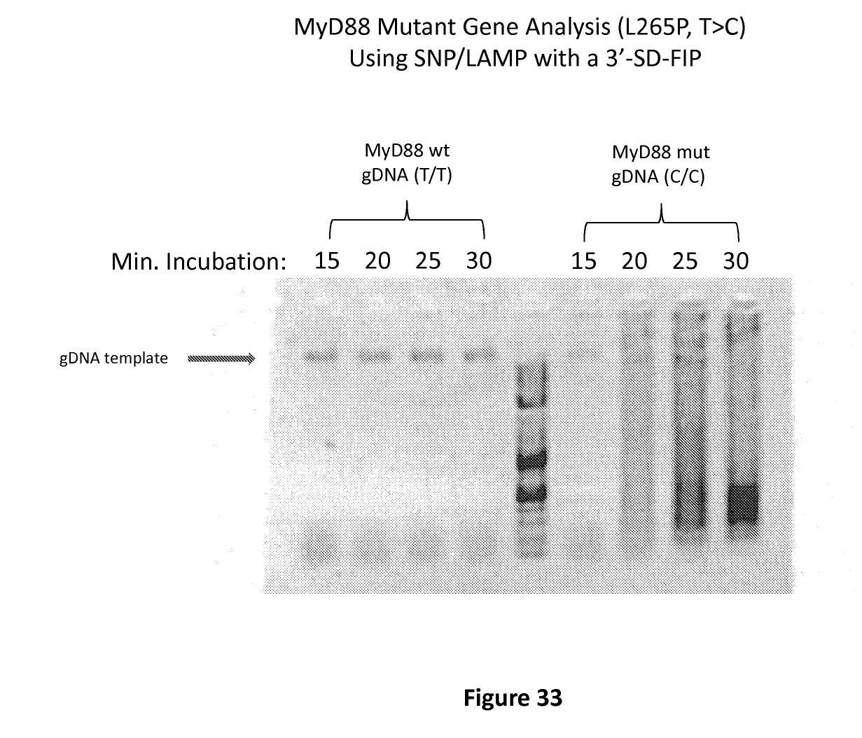

FIG. 33 depicts a SNP/LAMP amplicon designed for a mutation (C/C) that is frequently seen in the MyD88 gene (L265P, T>C) of patients with ABC-DLBCL (activated B-cell-like (ABC) subtype of diffuse large B-cell lymphoma (DLBCL)). This panel demonstrates a time-course looking at gDNA from a cell line homozygous (C/C) for the mutation. In this example, even with high concentrations of wild type (T/T) gDNA (˜25 ng), no amplification is seen over the duration of the experiment, whereas, the mutant gDNA shows a visible product at 20 minutes. Again this result shows the predicted outcome. Reactions were run at 60° C. with only FIP and BIP primers, with FIP being the SNP discriminator.

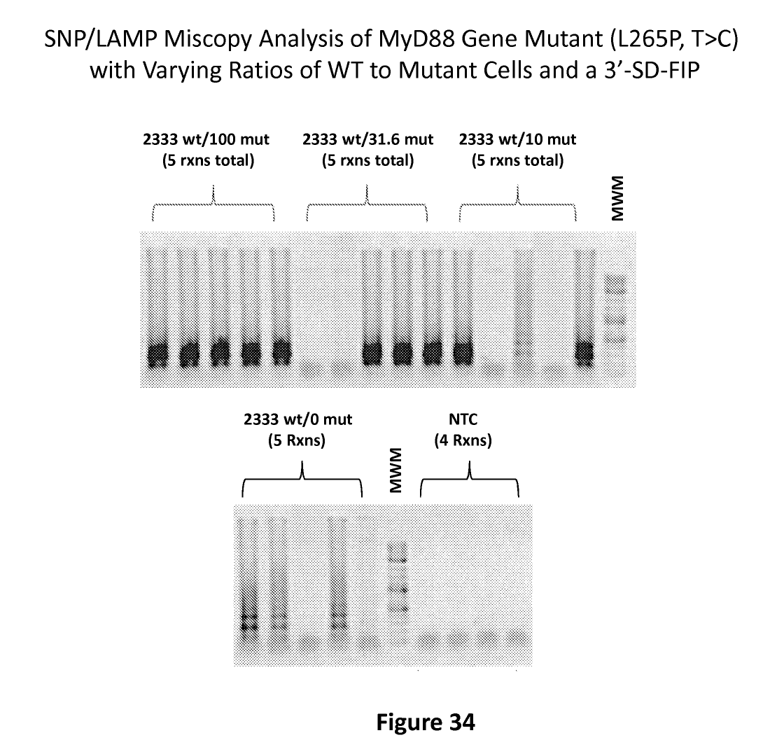

FIG. 34 depicts the results of a study to determine the sensitivity and specificity of the MyD88 C/C amplicon. This was carried out by mixing a cell-gDNA equivalent of the wild type (T/T) cells with decreasing amounts of the cell-gDNA equivalent of the mutant (C/C) cells; reaction mixtures assume 6 pg of gDNA per cell. At a ratio of 2333 wt cells/100 mutant cells, all 5 reactions are strongly positive, but the strong signals fallout sporadically as the amount of mutant gDNA is reduced. Unfortunately, but highly relevant to the miscopy phenomena, even when no mutant gDNA is present, moderate to weak products are seen in 4 out of 5 reactions, while no products are seen in the NTCs. Here again, miscopy is potentially confounding the interpretation of the results. Reactions were run at 30 min at 60° C. and in this case include forward and reverse outer primers.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A buffer for isothermic amplification of nucleic acid, the buffer comprising:
45 mM Tris-HCl at pH 7.75-8.0;
25 mM KCl;
25 mM (NH₄)₂SO₄;
0.2-0.25 mM dNTP;
1-8 units Bst DNA polymerase, large fragment;
550-825 nM Forward Inner Primer (FIP); and
550-825 nM Backward Inner Primer (BIP).

2. The buffer of claim 1, additionally comprising 4 mM MgSO₄.

3. The buffer of claim 1, additionally comprising 3 mM MgCl₂.

4. The buffer of claim 1, additionally comprising an enhancer.

5. The buffer of claim 2, wherein the enhancer is 2%-4% DMSO.

6. The buffer of claim 2, wherein the enhancer is a 1× solution of 0.6M betaine and 2% DMSO.

7. The buffer of claim 6, wherein the 1× solution of 0.6M betaine and 2% DMSO is added at 0.5×.

8. A method of performing a modified LAMP reaction, the method comprising:
preparing on ice a reaction mixture comprising target nucleic acid and 1× buffer of claim 1;
heating the reaction mixture at 60° C.; and
returning the reaction mixture to ice;
detecting the modified LAMP reaction products.

9. The method of performing a modified LAMP reaction of claim 8, wherein the reaction mixture is heated for 15-20 minutes.

10. The method of performing a modified LAMP reaction of claim 8, wherein the reaction mixture additionally comprises a SNP-discriminating forward loop primer (SD-LP).

11. The method of performing a modified LAMP reaction of claim 8, wherein the modified LAMP reaction products are detected by fluorescence.

12. The method of performing a modified LAMP reaction of claim 8, wherein the reaction mixture additionally comprises one or more primers selected from the group consisting of a back loop primer (BLP) and a forward loop primer (FLP).

13. A kit for performing isothermic amplification of nucleic acid, said kit comprising a composition containing the buffer of claim 1.

14. The kit of claim 13, wherein the kit further comprises instructional material for performing the method of the modified LAMP reaction of claim 8.
